(19) United States
(12) Patent Application Publication
(10) Pub. No.: US 2020/0270216 A1
(43) Pub. Date: Aug. 27, 2020

(54) COMPOSITIONS AND METHODS FOR INHIBITING N-SMASE2

(71) Applicants: Tina BILOUSOVA, Santa Monica, CA (US); Barbara JAGODZINSKA, Redwood City, CA (US); Varghese JOHN, Los Angeles, CA (US); The Regents of the University of California, Oakland, CA (US)

(72) Inventors: Tina Bilousova, Los Angeles, CA (US); Barbara Jagodzinska, Los Angeles, CA (US); Varghese John, Los Angeles, CA (US)

(21) Appl. No.: 16/647,364

(22) PCT Filed: Sep. 14, 2018
(86) PCT No.: PCT/US18/51156

§ 371 Date: Mar. 13, 2020

Related U.S. Application Data
(60) Provisional application No. 62/559,209, filed on Sep. 15, 2017.
(60) Provisional application No. 62/650,643, filed on Mar. 30, 2018.

(51) Int. Cl. C07D 239/56; C07D 239/54; C07D 401/12; C07D 239/36; C07D 405/06; A61P 25/16; A61P 25/28

(52) U.S. Cl. CPC C07D 239/56; C07D 239/54; C07D 401/12; A61P 25/28; C07D 405/06; A61P 25/16; C07D 239/36

(57) ABSTRACT

Provided herein are compounds of Formula (I) and (II) and their salts, and compositions comprising such compounds that are useful for useful for modulating neutral sphingomyelinase 2 (n-SMase2) in cells. Also disclosed herein are methods of using the disclosed compounds and compositions for inhibiting the spread of Tau seeds from donor cells to recipient cells. Moreover, disclosed herein are methods of using the disclosed compounds and compositions for treating or preventing a neurodegenerative disorder, such as a tauopathy, Alzheimer's disease (AD), Parkinson's disease (PD), Huntington's disease (HD), Lewy body dementia, frontotemporal dementia, and amyotrophic lateral sclerosis (ALS).

### RELATED APPLIACTIONS

This application claims the benefit of U.S. Provisional Patent Application No. 62/559,209, filed on Sep. 15, 2017, and U.S. Provisional Patent Application No. 62/650,643, filed on Mar. 30, 2018. The contents of each of these applications is hereby incorporated by reference in its entirety.

### STATEMENT OF GOVERNMENT SUPPORT

This invention was made with government support under Grant Number 1464898, awarded by the National Science Foundation. The government has certain rights in the invention.

### BACKGROUND

A neurodegenerative disease is an umbrella term for the progressive degeneration of neurons in, e.g., the central nervous system (CNS), characterized by molecular and genetic changes in nerve cells that result in nerve cell degeneration and ultimately nerve dysfunction and death. Neurodegenerative diseases affect an estimated 50 million Americans each year, exacting an incalculable personal toll and an annual economic cost of hundreds of billions of dollars in medical expenses and lost productivity. Neurodegenerative diseases include, but are not limited to, tauopathies, Alzheimer's disease (AD), amyotrophic lateral sclerosis (ALS), Huntington's disease (HD), and Parkinson's disease (PD).

At present, there are no effective treatments for halting, preventing, or reversing the progression of such neurodegenerative diseases. Therefore, there is an urgent need for pharmaceutical agents capable of slowing the progression of the aforementioned neurodegenerative diseases (and others) and/or preventing them in the first place.

### SUMMARY

Provided herein are compounds and compositions useful for modulating neutral sphingomyelinase 2 (n-SMase2) activity in cells. Also disclosed herein are methods of using the disclosed compounds and compositions for inhibiting the spread of Tau seeds from donor cells to recipient cells. Moreover, disclosed herein are methods of using the disclosed compounds and compositions for treating or preventing a neurodegenerative disorder, such as a tauopathy, Alzheimer's disease (AD), Parkinson's disease (PD), Huntington's disease (HD), Lewy body dementia, frontotemporal dementia, or amyotrophic lateral sclerosis (ALS).

Provided herein are compounds having a structure of Formula (I):

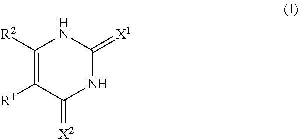

or a pharmaceutically acceptable salt thereof, wherein:
X¹ and X² are each independently O or S; and
R¹ and R² are each, independently alkyl, —C(O)-alkyl, haloalkyl, alkoxy, cycloalkyl, aryl, —C(O)-aryl, aralkyl, or haloalkyl, each of which is unsubstituted or substituted with one or more substituents.

Also provided herein are compounds having a structure of Formula (II):

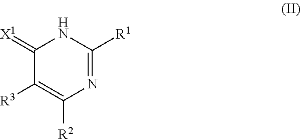

or a pharmaceutically acceptable salt thereof, wherein:
X¹ is O or S;
R¹ is alkyl, alkenyl, aryl, aralkyl, heteroaryl, —O-alkyl, —O-alkenyl, —O-haloalkyl, —O-cycloalkyl, —O-aralkyl, —O-aralkoxy, —O-aryl, —O-heteroaryl, —S-alkyl, —S-alkenyl, —S-haloalkyl, —S-cycloalkyl, —S-aralkyl, —S-aralkoxy, —S-aryl, or —S-heteroaryl, each of which is unsubstituted or substituted with one or more substituents; and
R² and R³ are each, independently CN, C(O)-alkyl, alkyl, haloalkyl, alkoxy, cycloalkyl, aryl, aralkyl, alkyl-O-aryl, or heteroaryl, wherein each of —C(O)-alkyl, alkyl, haloalkyl, alkoxy, cycloalkyl, aryl, aralkyl, aryl-O-alkyl, or heteroaryl is unsubstituted or substituted with one or more substituents.

In one aspect, the invention provides pharmaceutical compositions of compounds disclosed herein, e.g., that comprise a pharmaceutically acceptable excipient and a compound of Formula (I) or (II), or a pharmaceutically acceptable salt thereof. The pharmaceutical compositions can be used in therapy, e.g., for treating a disease or condition disclosed herein in a subject.

In another aspect, provided herein are methods for modulating neutral sphingomyelinase 2 (n-SMase2) in a cell, comprising contacting a cell with a compound of Formula (I) or (II), or a pharmaceutically acceptable salt thereof, or a pharmaceutical composition as disclosed herein. In some embodiments, the cell occurs in a subject, and the method serves to treat an n-SMase2-mediated condition and/or disease.

In yet another aspect, provided herein are methods for inhibiting the spread of Tau seeds from donor cells to recipient cells, comprising contacting the cells with a compound of Formula (I) or (II), or a pharmaceutically acceptable salt thereof, or a pharmaceutical composition as disclosed herein. In some embodiments, the cell occurs in a subject, and the method serves to treat a condition and/or disease associated with Tau deposits.

In still another aspect, provided herein are method for treating or preventing a disease or disorder associated with accumulation and/or aggregation of misfolded proteins, comprising administering to a subject in need thereof a therapeutically effective amount of a compound of Formula (I) or (II), or a pharmaceutically acceptable salt thereof, or a pharmaceutical composition as disclosed herein.

In another aspect, provided herein are methods for treating or preventing a neurodegenerative disease or disorder, comprising administering to a subject in need thereof a therapeutically effective amount of a compound of Formula (I) or (II), or a pharmaceutically acceptable salt thereof, or a pharmaceutical composition as disclosed herein. In some embodiments, the neurodegenerative disorder is selected from a tauopathy, Alzheimer's disease, Parkinson's disease, Huntington's disease, Lewy body dementia, frontotemporal dementia, and amyotrophic lateral sclerosis.

### BRIEF DESCRIPTION OF THE DRAWINGS

FIG. 1A depicts that modeling shows cambinol is predicted to bind and modulate the DK-switch in the active site domain (arrows).

FIG. 1B depicts that n-SMase2 residues are involved in interaction with cambinol.

FIG. 1C depicts the binding free energy calculations after simulation of cambinol binding to the DK-switch region of n-SMase2.

FIG. 2 depicts dose response curve showing the inhibition of n-SMase2 activity in the presence of cambinol.

FIG. 3A depicts tau biosensor cells that were seeded with AD human brain derived synaptosomal (P2) extract and grow in presence of 10 μM of n-SMase2 inhibitor DDL-112 or corresponded amount of DMSO. Decrease in the tau aggregation in donor cells in the presents of cambinol suggested either inhibition of tau seed contained EVs or inhibition of tau aggregation by cambinol.

FIG. 3B depicts cells that were imaged in 24 hours after seeding. After 60 hours cell culture medium and cells were collected. Cells were fixed and FRET signal was analyzed using flow cytometry.

FIG. 3C depicts Cell culture medium was used for extracellular vesicle (EV) purification using ExoQuick methods. Amount of exosomal marker (CD63) in the purified EV fractions was assessed by Western blotting analysis.

FIG. 3D depicts nSMase2 activity in Tau biosensor cell homogenate inhibited by cambinol (IC₅₀˜14 uM).

FIG. 4A depicts the “D+R approach” scheme.

FIG. 4B depicts Image stream imaging flow cytometry for D and R tau biosensor cells.

FIG. 4C depicts bar graphs of high throughput flow cytometry: two unrelated n-SMase2 inhibitors limit tau seed transmission in D+R model system.

FIG. 4D depicts dose-response curve for inhibition of tau seed transfer by cambinol.

FIG. 5A depicts the “EV-mediated transfer” and “D+R” approach scheme.

FIG. 5B depicts that Cambinol inhibits nSMase2 activity in cell extracts and EV-mediated tau seed propagation.

FIG. 5C depicts an electron microscopy image of purified EV.

FIG. 5D depicts a nano tracker analysis data for EVs.

FIG. 5E depicts the WB analysis of purified EVs.

FIG. 5F depicts the densitometry results for WB analysis.

FIG. 6A depicts pharmacokinetic (PK) analysis.

FIG. 6B depicts the target-engagement (n-SMase 2 inhibition in brain homogenate) study in mice with DDL-112.

FIG. 7A depicts the initial in vivo proof-of-efficacy using DDL-112 in Thy-1 aSyn (alpha-synuclein) mice treated with DDL-112 (cambinol) at 100 mg/kg by the oral route. The data shows a trend to decrease in the aSyn levels in the substantia nigra (SN) a key region affected in Parkinson's disease.

FIG. 7B depicts tyrosine hydroxylase (TH) levels in SN that were also measured.

### DETAILED DESCRIPTION

Accumulation and aggregation of misfolded proteins is a common feature of a wide group of neurodegenerative disorders, including Alzheimer's disease, Parkinson's disease, Lewy body dementia, frontotemporal dementia, and amyotrophic lateral sclerosis. Recent data suggests a “prion-like” mode of the pathology propagation in the aforementioned disorders: misfolded disease-specific proteins, released from donor cells, can be internalized by recipient cells, where they play a role of proteopathic seeds by templating abnormal protein conformations.

Misfolded/aggregate-prone proteins are often found in extracellular vesicles (EVs) purified from blood and cerebrospinal fluid of patients with neurodegenerative disorders and in animal models. Since EVs can shuttle between cells, they are good candidates for being a part of the proteopathic seed delivery system. Exosomes are small EVs (30 to 130 nm in diameter), which originate from a specific type of late endosomes—multivesicular bodies (MVBs), and are then released to extracellular space upon MVB fusion with plasma membrane.

Inhibition of neutral sphingomyelinase 2 (n-SMase2), the gatekeeping enzyme of ceramide-mediated exosome production, has been found beneficial in animal models of primary tauopathy, Alzheimer's disease, and Lewy body dementia. n-SMase2 is highly expressed in the brain and its activity is upregulated with age. Moreover, brain ceramide level has been found to be elevated in different neurodegenerative disorders. Thus, modulation of n-SMase2 activity represents a promising strategy to control exosome-mediated proteopathic seed propagation.

Accordingly, this invention provides, inter alia, compounds and methods for modulating n-SMase2. Compounds disclosed herein may be provided as pharmaceutical compositions. Such pharmaceutical compositions may further comprise one or more pharmaceutically acceptable carriers.

The compounds and pharmaceutical compositions disclosed herein are useful for treating various diseases and conditions, such as disorders related to neurodegeneration, tauopathies, Alzheimer's disease, Parkinson's disease, Huntington's disease, Lewy body dementia, frontotemporal dementia, and amyotrophic lateral sclerosis (ALS).

### Definitions

The term “alkyl” used alone or as part of a larger moiety, such as “alkoxy”, “haloalkyl”, “cycloalkyl”, “heterocycloalkyl”, and the like, refers to a straight chained or branched non-aromatic hydrocarbon which is completely saturated. Typically, a straight chained or branched alkyl group has from 1 to about 20 carbon atoms, preferably from 1 to about 10 carbon atoms, unless otherwise defined. Examples of straight chained and branched alkyl groups include, but are not limited to, methyl, ethyl, propyl, isopropyl, n-butyl, sec-butyl, tert-butyl, n-pentyl, n-hexyl, n-heptyl, and n-octyl. A C₁-C₆ straight chained or branched alkyl group is also referred to as a “lower alkyl” group.

Moreover, the term “alkyl” (or “lower alkyl”), as used herein, is intended to include both “unsubstituted alkyls” and “substituted alkyls”, the latter of which refers to alkyl moieties having substituents replacing a hydrogen on one or more carbons of the hydrocarbon backbone. Such substituents, if not otherwise specified, can include, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. The skilled artisan will understand that the moieties substituted on the hydrocarbon chain can themselves be substituted, if appropriate. For example, the substituents of a substituted alkyl may include substituted and unsubstituted forms of amino, azido, imino, amido, phosphoryl (including phosphonate and phosphinate), sulfonyl (including sulfate, sulfonamido, sulfamoyl and sulfonate), and silyl groups, as well as ethers, alkylthios, carbonyls (including ketones, aldehydes, carboxylates, and esters), —CF₃, —CN and the like. Exemplary substituted alkyls are described below. Cycloalkyls can be further substituted with alkyls, alkenyls, alkoxys, alkylthios, aminoalkyls, carbonyl-substituted alkyls, —CF₃, —CN, and the like.

The term “alkenyl”, as used herein, refers to an aliphatic group containing at least one double bond and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed above, except where stability is prohibitive. For example, substitution of alkenyl groups by one or more alkyl, carbocyclyl, aryl, heterocyclyl, or heteroaryl groups is contemplated.

The term “Cₓ-C_{y}” when used in conjunction with a chemical moiety, such as, for example, acyl, acyloxy, alkyl, alkenyl, alkynyl, or alkoxy is meant to include groups that contain from x to y carbons in the chain, wherein “x” and “y” are integers selected from 1 to about 20, and wherein x is an integer of lesser value than y, and x and y are not the same value. For example, the term “Cₓ-C_{y}-alkyl” refers to substituted or unsubstituted saturated hydrocarbon groups, including straight-chain alkyl and branched-chain alkyl groups that contain from x to y number of carbons in the chain, including haloalkyl groups such as trifluoromethyl and 2,2,2-tirfluoroethyl, etc. The terms “C₂-C_{y}-alkenyl” and “C₂-C_{y}-alkynyl” refer to substituted or unsubstituted unsaturated aliphatic groups analogous in length and possible substitution to the alkyls described above, but that contain at least one double or triple bond respectively. As applied to haloalkyls, “Cₓ-C_{y}” indicates that the group contains from x to y number of carbons and heteroatoms in the chain. As applied to carbocyclic structures, such as aryl and cycloalkyl groups, “Cₓ-C_{y}” indicates that the ring comprises x to y number of carbon atoms in the ring. As applied to heterocyclic structures, such as heteroaryl and heterocyclyl groups, “Cₓ-C_{y}” indicates that the ring contains from x to y carbons in the ring.

The term “alkoxy” refers to an alkyl group, preferably a lower alkyl group, having an oxygen attached thereto. Representative alkoxy groups include methoxy, ethoxy, propoxy, tert-butoxy and the like.

The terms “haloalkyl” and “haloalkoxy” refer to alkyl or alkoxy, as the case may be, substituted with one or more halogen atoms. In some embodiments, “haloalkyl” and “haloalkoxy” refer to alkyl or alkoxy, as the case may be, substituted with one or more fluorine, chlorine, bromine, or iodine atoms.

The term “halogen” refers to fluorine or fluoro (F), chlorine or chloro (Cl), bromine or bromo (Br), or iodine or iodo (I).

The term “cycloalkyl”, as used herein, refers a substituted or unsubstituted cyclic hydrocarbon which is completely saturated. Cycloalkyl includes monocyclic and bicyclic rings. Typically, a monocyclic cycloalkyl group has from 3 to about 10 carbon atoms, more typically 3 to 8 carbon atoms, unless otherwise defined. The second ring of a bicyclic cycloalkyl may be selected from saturated, unsaturated, and aromatic rings. Cycloalkyl includes bicyclic molecules in which one, two, or three or more atoms are shared between the two rings. The term “fused cycloalkyl” refers to a bicyclic cycloalkyl in which each of the rings shares two adjacent atoms with the other ring. The second ring of a fused bicyclic cycloalkyl may be selected from saturated, unsaturated and aromatic rings. A “cycloalkenyl” group is a cyclic hydrocarbon containing one or more double bonds.

The term “aralkyl”, as used herein, refers to an alkyl group substituted with an aryl group.

The term “aralkoxy”, as used herein, refers to an alkoxy group substituted with an aryl group.

The term “aryl”, as used herein, refers to substituted or unsubstituted single-ring aromatic groups in which each atom of the ring is carbon. Preferably, the ring is a 5- to 7-membered ring, more preferably a 6-membered ring. The term “aryl” also includes polycyclic ring systems having two or more cyclic rings in which two or more carbons are common to two adjoining rings wherein at least one of the rings is aromatic, e.g., the other cyclic rings can be cycloalkyls, cycloalkenyls, cycloalkynyls, aryls, heteroaryls, and/or heterocyclyls. Aryl groups include benzene, naphthalene, phenanthrene, phenol, aniline, and the like.

“The term “heterocyclyl” and “heterocycle”, as used herein, refer to substituted or unsubstituted non-aromatic ring structures, preferably 3- to 10-membered rings, more preferably 3- to 7-membered rings, whose ring structures include at least one heteroatom, preferably one to four heteroatoms, more preferably one or two heteroatoms. Such heterocycles also include polycyclic ring systems having two or more cyclic rings in which two or more carbons are common to two adjoining rings wherein at least one of the rings is heterocyclic, e.g., the other cyclic rings can be cycloalkyls, cycloalkenyls, cycloalkynyls, aryls, heteroaryls, and/or heterocyclyls. Heterocyclyl groups include, for example, piperidine, piperazine, pyrrolidine, morpholine, lactones, lactams, and the like.

The term “heteroaryl” includes substituted or unsubstituted aromatic single ring structures, preferably 5- to 7-membered rings, more preferably 5- to 6-membered rings, whose ring structures include at least one heteroatom, preferably one to four heteroatoms, more preferably one or two heteroatoms. The terms “heteroaryl” and “hetaryl” also include polycyclic ring systems having two or more cyclic rings in which two or more carbons are common to two adjoining rings wherein at least one of the rings is heteroaromatic, e.g., the other cyclic rings can be cycloalkyls, cycloalkenyls, cycloalkynyls, aryls, heteroaryls, and/or heterocyclyls. Heteroaryl groups include, for example, pyrrole, furan, thiophene, imidazole, oxazole, thiazole, pyrazole, pyridine, pyrazine, pyridazine, and pyrimidine, and the like.

The term “substituted” refers to moieties having substituents replacing a hydrogen on one or more carbons or heteroatoms of the moiety. The skilled artisan will understand that “substitution” or “substituted with” includes the implicit proviso that such substitution is in accordance with permitted valence of the substituted atom and the substituent, and that the substitution results in a stable compound, e.g., which does not spontaneously undergo transformation such as by rearrangement, cyclization, elimination, etc. As used herein, the term “substituted” is contemplated to include all permissible substituents of organic compounds.

In some embodiments, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this invention, the heteroatoms such as nitrogen may have hydrogen substituents and/or any permissible substituents of organic compounds described herein which satisfy the valences of the heteroatoms. Substituents can include any substituents described herein, such as, for example, a halogen, a hydroxyl, a carbonyl (such as a carboxyl, an alkoxycarbonyl, a formyl, or an acyl), a thiocarbonyl (such as a thioester, a thioacetate, or a thioformate), an alkoxyl, a phosphoryl, a phosphate, a phosphonate, a phosphinate, an amino, an amido, an amidine, an imine, a cyano, a nitro, an azido, a sulfhydryl, an alkylthio, a sulfate, a sulfonate, a sulfamoyl, a sulfonamido, a sulfonyl, a heterocyclyl, an aralkyl, or an aromatic or heteroaromatic moiety. The skilled artisan will understand that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

In certain embodiments, compounds of the invention may be racemic. In certain embodiments, compounds of the invention may be enriched in one enantiomer. For example, a compound of the invention may have greater than about 30% ee, about 40% ee, about 50% ee, about 60% ee, about 70% ee, about 80% ee, about 90% ee, or even about 95% or greater ee. In certain embodiments, compounds of the invention may have more than one stereocenter. In certain such embodiments, compounds of the invention may be enriched in one or more diastereomer. For example, a compound of the invention may have greater than about 30% de, about 40% de, about 50% de, about 60% de, about 70% de, about 80% de, about 90% de, or even about 95% or greater de.

In certain embodiments, the therapeutic preparation may be enriched to provide predominantly one enantiomer of a compound (e.g., a compound of Formula (I) or (II)). An enantiomerically enriched mixture may comprise, for example, at least about 60 mol percent of one enantiomer, or more preferably at least about 75, about 90, about 95, or even about 99 mol percent. In certain embodiments, the compound enriched in one enantiomer is substantially free of the other enantiomer, wherein substantially free means that the substance in question makes up less than about 10%, or less than about 5%, or less than about 4%, or less than about 3%, or less than about 2%, or less than about 1% as compared to the amount of the other enantiomer, e.g., in the composition or compound mixture. For example, if a composition or compound mixture contains about 98 grams of a first enantiomer and about 2 grams of a second enantiomer, it would be said to contain about 98 mol percent of the first enantiomer and only about 2% of the second enantiomer.

In certain embodiments, the therapeutic preparation may be enriched to provide predominantly one diastereomer of a compound (e.g., a compound of Formula (I) or (II)). A diastereomerically enriched mixture may comprise, for example, at least about 60 mol percent of one diastereomer, or more preferably at least about 75, about 90, about 95, or even about 99 mol percent.

The term “subject” to which administration is contemplated includes, but is not limited to, humans (i.e., a male or female of any age group, e.g., a pediatric subject (e.g., infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, quail, and/or turkeys. Preferred subjects are humans.

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

The term “treating” includes prophylactic and/or therapeutic treatments. The term “prophylactic or therapeutic” treatment is art-recognized and includes administration to the host of one or more of the subject compositions. If it is administered prior to clinical manifestation of the unwanted condition (e.g., disease or other unwanted state of the host animal) then the treatment is prophylactic (i.e., it protects the host against developing the unwanted condition), whereas if it is administered after manifestation of the unwanted condition, the treatment is therapeutic, (i.e., it is intended to diminish, ameliorate, or stabilize the existing unwanted condition or side effects thereof).

The term “neurodegeneration” refers broadly to a defect involving or relating to the nervous system. As used herein, the terms “neurodegenerative disorder” or “neurodegenerative disease” refer broadly to disorders or diseases that affect the nervous system, including but are not limited to tauopathies, Parkinson's disease (PD), Alzheimer's disease (AD), Huntington's disease, Lewy body demenia, Batten disease, frontotemporal dementia, amyotrophic lateral sclerosis (ALS), and the like.

The term “tauopathy” refers to a class of neurodegenerative diseases associated with the pathological aggregation of tau protein neurofibrillary or gliofibrillary tangles in the mammalian, e.g., human, brain. Tauopathies, i.e., conditions in which neurofibrillary tangles (NFT) are predominantly observed, include, but are not limited to, primary age-related tauopathy (PART); neurofibrillary tangle-predominant senile dementia; chronic traumatic encephalopathy (e.g., dementia pugilistica); progressive supranuclear palsy; corticobasal degeneration; parkinsonism linked to chromosome 17; Lytico-Bodig disease (i.e., Parkinson-dementia complex of Guam); ganglioglioma and gangliocytoma; meningioangiomatosis; postencephalitic parkinsonism; subacute sclerosing panencephalitis; lead encephalopathy; tuberous sclerosis; Hallervorden-Spatz disease; lipofuscinosis; Pick's disease and Pick's complex; corticobasal degeneration; and Argyrophilic grain disease (AGD).

The term “donor cells” refers to neuronal cells that are capable of releasing Tau seeds through exocytosis or through vesicles such as exosomes (derived from multivescular bodies (MVBs)), which can fuse with recipient neuronal cells (referred to herein as “recipient cells”).

In certain embodiments, compounds of Formula (I), or (II) may be used alone or conjointly administered with another type of therapeutic compound or agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the subject, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, or a week of one another. Thus, a subject who receives such treatment can benefit from a combined effect of different therapeutic compounds.

### Compounds of the Invention

Cambinol (Formula (A)) is a potent inhibitor of human n-SMase2, but its instability and low permeability into the blood-brain barrier make it a poor drug candidate.

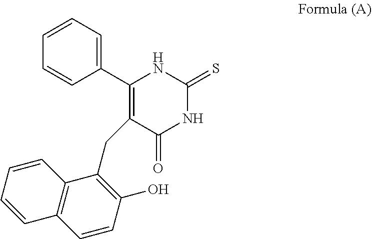 [SMILES: O=c1[nH]c(=S)[nH]c(-c2ccccc2)c1Cc1c(O)ccc2ccccc12]

Thus, provided herein are cambinol analogs as well as structurally unrelated compounds with superior potency and ADME (absorption/distribution/metabolism/excretion) characteristics.

Specifically, included in the present disclosure are compounds of Formula (I):

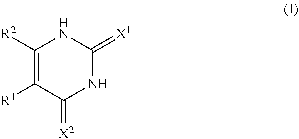

or a pharmaceutically acceptable salt thereof, wherein:
X¹ and X² are each independently O or S; and
R¹ and R² are each, independently alkyl, —C(O)-alkyl, haloalkyl, alkoxy, cycloalkyl, aryl, —C(O)-aryl, aralkyl, or haloalkyl, each of which is unsubstituted or substituted with one or more sub stituents.

In some embodiments, the compound of Formula (I) is not a compound of Formula (A):

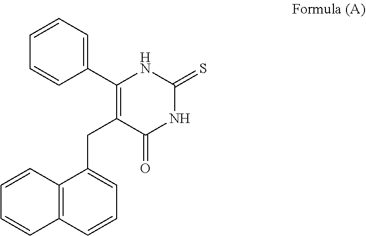 [SMILES: O=c1[nH]c(=S)[nH]c(-c2ccccc2)c1Cc1cccc2ccccc12]

or a pharmaceutically acceptable salt thereof.

In some embodiments, the compound of Formula (I) has a structure of Formula (Ia):

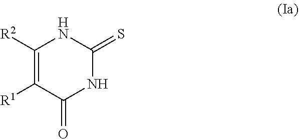

or a pharmaceutically acceptable salt thereof.

Alternatively, in some embodiments, the compound of Formula (I) has a structure of Formula (Ib):

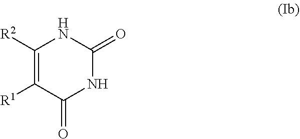

or a pharmaceutically acceptable salt thereof.

In certain embodiments of the compounds of Formula (I), (Ia), and (Ib), R¹ and R² are each, independently —C(O)—C₁-C₄-alkyl, —C₁-C₄-alkyl, aryl, —C(O)-aryl, or aralkyl, each of which is unsubstituted or substituted with one or more substituents. In some such embodiments, R¹ and R² are each, independently unsubstituted —C(O)—C₁-C₄-alkyl, unsubstituted —C₁-C₄-alkyl, unsubstituted aryl, unsubstituted —C(O)-aryl, or unsubstituted aralkyl. In some preferred embodiments, R¹ is phenyl. Alternatively, R¹ and R² are each, independently —C(O)—C₁-C₄-alkyl, —C₁-C₄-alkyl, aryl, —C(O)-aryl, or aralkyl, each of which is substituted with halogen, OH, —C₁-C₄-alkyl, —C₁-C₄-haloalkyl, —C₁-C₄-alkoxy, or —C₁-C₄-haloalkoxy.

In preferred embodiments, the compound of Formula (I) is selected from:

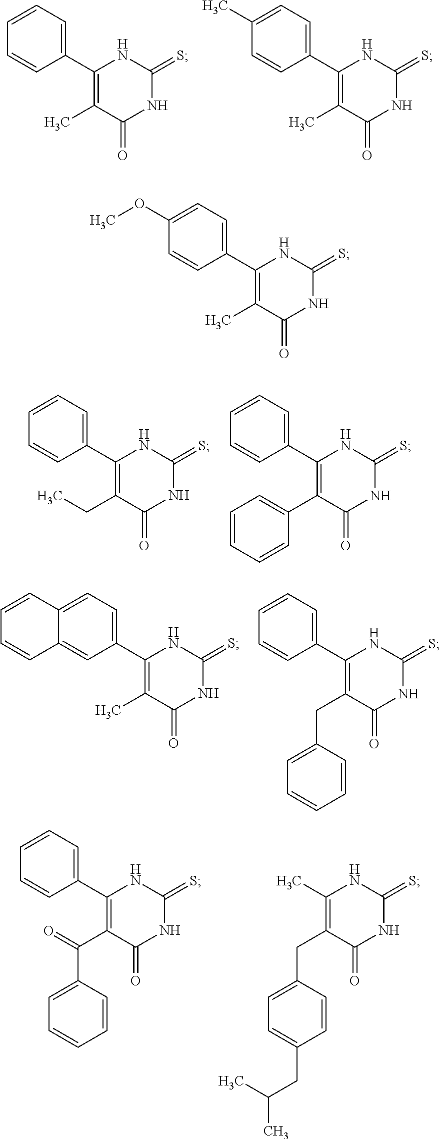

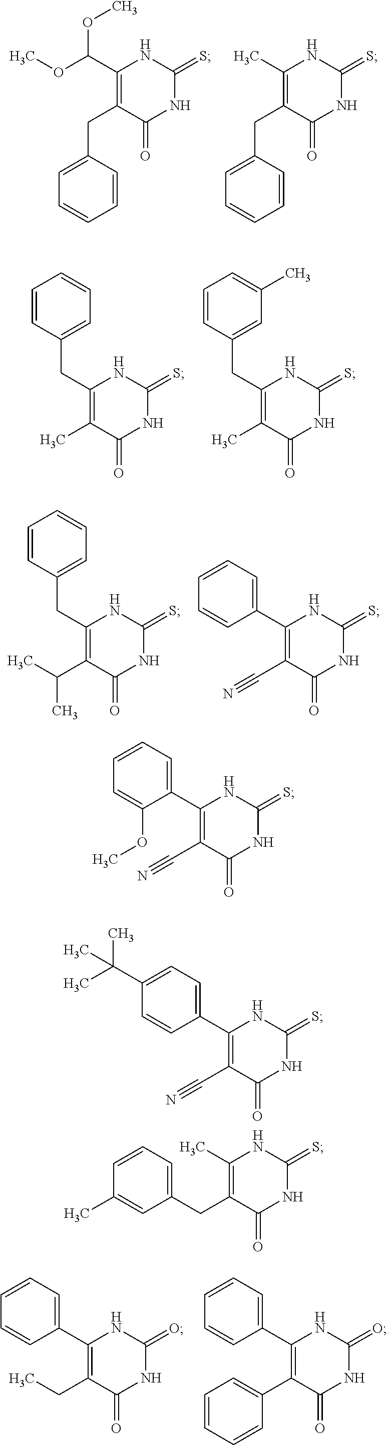

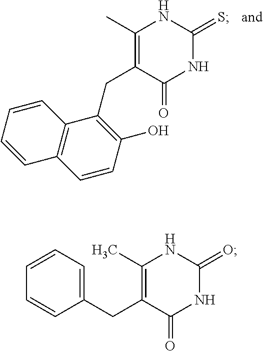 [SMILES: Cc1[nH]c(=S)[nH]c(=O)c1Cc1c(O)ccc2ccccc12 ; Cc1[nH]c(=O)[nH]c(=O)c1Cc1ccccc1]

or a pharmaceutically acceptable salt thereof.

Also included in the present disclosure are compounds of Formula (II):

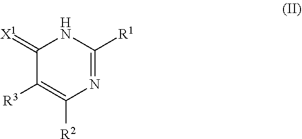

or a pharmaceutically acceptable salt thereof, wherein:
X¹ is O or S;
R¹ is alkyl, alkenyl, aryl, aralkyl, heteroaryl, —O-alkyl, —O-alkenyl, —O-haloalkyl, —O-cycloalkyl, —O-aralkyl, —O-aralkoxy, —O-aryl, —O-heteroaryl, —S-alkyl, —S-alkenyl, —S-haloalkyl, —S-cycloalkyl, —S-aralkyl, —S-aralkoxy, —S-aryl, or —S-heteroaryl, each of which is unsubstituted or substituted with one or more substituents; and
R² and R³ are each, independently CN, C(O)-alkyl, alkyl, haloalkyl, alkoxy, cycloalkyl, aryl, aralkyl, alkyl-O-aryl, or heteroaryl, wherein each of —C(O)-alkyl, alkyl, haloalkyl, alkoxy, cycloalkyl, aryl, aralkyl, aryl-O-alkyl, or heteroaryl is unsubstituted or substituted with one or more substituents.

In some embodiments, the compound of Formula (II) has a structure of Formula (IIa):

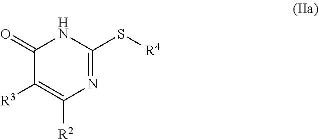

or a pharmaceutically acceptable salt thereof, wherein R⁴ is alkyl, alkenyl, haloalkyl, cycloalkyl, aralkyl, aralkoxy, aryl, or heteroaryl, each of which is unsubstituted or substituted with one or more substituents.

Alternatively, the compound of Formula (II) has a structure of Formula (IIb):

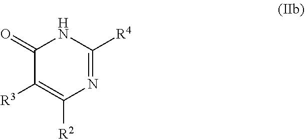

or a pharmaceutically acceptable salt thereof, wherein R⁴ is alkyl, alkenyl, haloalkyl, cycloalkyl, aralkyl, alkyl-O-aryl, aryl, or heteroaryl, each of which is unsubstituted or substituted with one or more substituents.

In some embodiments of the compounds of Formula (II), (IIa), and (IIb), R² is —C₁-C₄-alkyl, aryl, or aralkyl, each of which is unsubstituted or substituted with one or more substituents. In some such embodiments, R² is unsubstituted —C₁-C₄-alkyl, unsubstituted aryl, or unsubstituted aralkyl. Alternatively, R² is C₁-C₄-alkyl, aryl, or aralkyl, each of which is substituted with halogen, —OH, —C₁-C₄-alkyl, —C₁-C₄-haloalkyl, —C₁-C₄-alkoxy, or —C₁-C₄-haloalkoxy.

In some embodiments of the compounds of Formula (II), (IIa), and (IIb), R³ is —CN, —C(O)—C₁-C₄-alkyl, —C₁-C₆-alkyl, aryl, or aralkyl, wherein —C(O)—C₁-C₄-alkyl, —C₁-C₆-alkyl, aryl, or aralkyl is unsubstituted or substituted with one or more substituents. In some such embodiments, R³ is CN, unsubstituted —C(O)—C₁-C₄-alkyl, unsubstituted C₁-C₆-alkyl, unsubstituted aryl, or unsubstituted aralkyl. Alternatively, R³ is —C(O)—C₁-C₄-alkyl, —C₁-C₆-alkyl, aryl, or aralkyl, each of which is substituted with halogen, OH, —C₁-C₄-alkyl, —C₁-C₄-haloalkyl, —C₁-C₄-alkoxy, or —C₁-C₄-haloalkoxy.

In some embodiments of the compounds of Formula (II), (IIa), and (IIb), R⁴ is —C₁-C₄-alkyl, —C₂-C₄-alkenyl, aryl, aralkyl, or aryl-O—C₁-C₄-alkyl, each of which is unsubstituted or substituted with one or more substituents. In some such embodiments, R⁴ is unsubstituted —C₁-C₄-alkyl, unsubstituted —C₂-C₄-alkenyl, unsubstituted aryl, unsubstituted aralkyl, or unsubstituted aryl-O—C₁-C₄-alkyl. Alternatively, R⁴ is —C₁-C₄-alkyl, —C₂-C₄-alkenyl, aryl, aralkyl, or aryl-O—C₁-C₄-alkyl, each of which is unsubstituted or substituted with halogen, —OH, —C₁-C₄-alkyl, —C₁-C₄-haloalkyl, —C₁-C₄-alkoxy, or —C₁-C₄-haloalkoxy. In other embodiments, R⁴ is heteroaralkyl.

In preferred embodiments, the compound of Formula (II) is selected from:

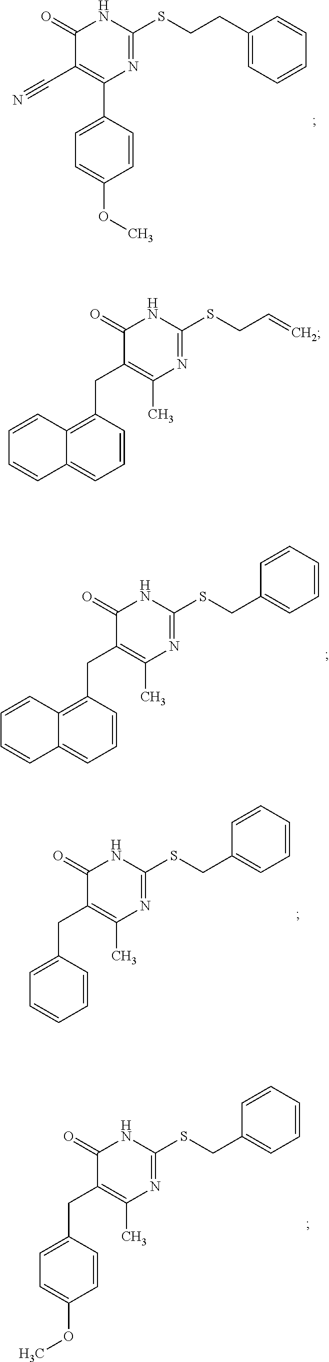 [SMILES: COc1ccc(-c2nc(SCCc3ccccc3)[nH]c(=O)c2C#N)cc1 ; C=CCSc1nc(C)c(Cc2cccc3ccccc23)c(=O)[nH]1 ; Cc1nc(SCc2ccccc2)[nH]c(=O)c1Cc1cccc2ccccc12 ; Cc1nc(SCc2ccccc2)[nH]c(=O)c1Cc1ccccc1 ; COc1ccc(Cc2c(C)nc(SCc3ccccc3)[nH]c2=O)cc1]

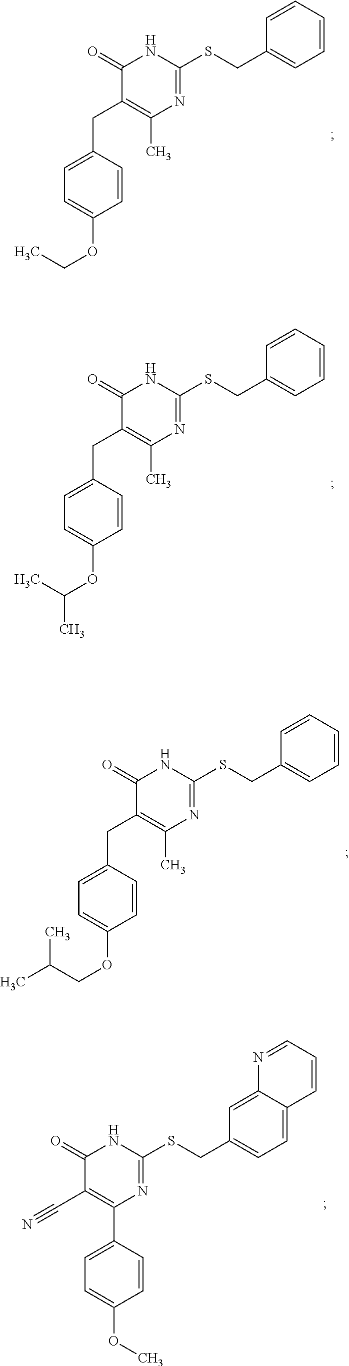 [SMILES: CCOc1ccc(Cc2c(C)nc(SCc3ccccc3)[nH]c2=O)cc1 ; Cc1nc(SCc2ccccc2)[nH]c(=O)c1Cc1ccc(OC(C)C)cc1 ; Cc1nc(SCc2ccccc2)[nH]c(=O)c1Cc1ccc(OCC(C)C)cc1 ; COc1ccc(-c2nc(SCc3ccc4cccnc4c3)[nH]c(=O)c2C#N)cc1]

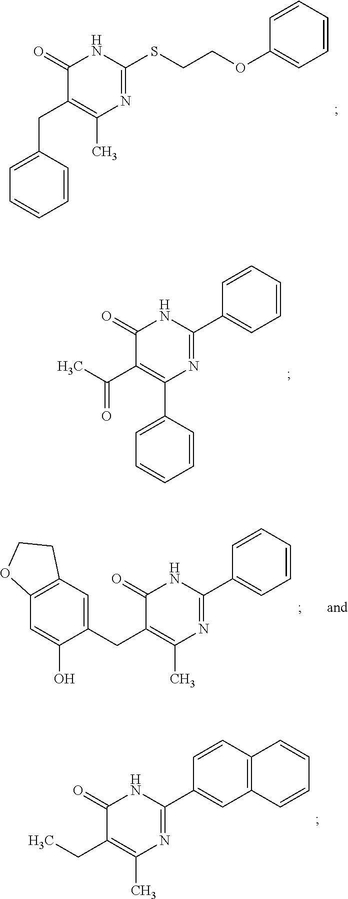 [SMILES: Cc1nc(SCCOc2ccccc2)[nH]c(=O)c1Cc1ccccc1 ; CC(=O)c1c(-c2ccccc2)nc(-c2ccccc2)[nH]c1=O ; Cc1nc(-c2ccccc2)[nH]c(=O)c1Cc1cc2c(cc1O)OCC2 ; CCc1c(C)nc(-c2ccc3ccccc3c2)[nH]c1=O]

or a pharmaceutically acceptable salt thereof.

### Methods of Treatment

Provided herein are methods for using the disclosed compounds and pharmaceutical compositions thereof. The disclosed compounds and pharmaceutical compositions thereof can be useful for a variety of therapeutic applications including, for example, treating and/or reducing a wide variety of diseases and disorders including, for example, diseases or disorders related to neurodegeneration. The methods comprise administering to a subject in need thereof a disclosed compound and/or pharmaceutical composition thereof.

In certain embodiments, the disclosed compounds and pharmaceutical compositions thereof may be used to modulate neutral sphingomyelinase 2 (n-SMase2) in a cell. In some such embodiments, the cells occur in a subject in need thereof, thereby treating an n-SMase2-mediated condition and/or disease, e.g., a neurodegenerative disease, e.g., a tauopathy, Alzheimer's disease, Parkinson's disease, Huntington's disease, Lewy body dementia, frontotemporal dementia, and amyotrophic lateral sclerosis (ALS).

In some embodiments, the disclosed compounds and pharmaceutical compositions thereof may be used to inhibit the spread of Tau seeds from donor cells to recipient cells. In some such embodiments, the cells occur in a subject in need thereof, thereby treating a condition and/or disease associated with Tau deposits, e.g., a neurodegenerative disease, e.g., a tauopathy, Alzheimer's disease, Parkinson's disease, Huntington's disease, Lewy body dementia, frontotemporal dementia, and amyotrophic lateral sclerosis (ALS).

In other embodiments, the disclosed compounds and pharmaceutical compositions thereof may be used to treat or prevent a disease or disorder associated with accumulation and/or aggregation of misfolded proteins, e.g., a neurodegenerative disease, e.g., a tauopathy, Alzheimer's disease, Parkinson's disease, Huntington's disease, Lewy body dementia, frontotemporal dementia, and amyotrophic lateral sclerosis (ALS).

### Pharmaceutical Compositions and Administration Thereof

The compositions and methods disclosed herein may be utilized to treat an individual in need thereof. In certain embodiments, the individual is a mammal such as a human, or a non-human mammal. When administered to an animal, such as a human, the composition or the compound is preferably administered as a pharmaceutical composition comprising, for example, a disclosed compound and a pharmaceutically acceptable carrier.

Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule (including sprinkle capsule and gelatin capsule), granule, lyophile for reconstitution, powder, solution, syrup, suppository, injection, or the like. The composition can also be present in a transdermal delivery system, e.g., a skin patch. The composition can also be present in a solution suitable for topical administration, such as an ointment or cream.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a selfemulsifying drug delivery system or a selfmicroemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example, orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin, or as an eye drop). The compound may also be formulated for inhalation. In certain embodiments, a compound may be simply dissolved or suspended in sterile water. Details of appropriate routes of administration and compositions suitable for same can be found in, for example, U.S. Pat. Nos. 6,110,973, 5,763,493, 5,731,000, 5,541,231, 5,427,798, 5,358,970 and 4,172,896, as well as in patents cited therein.

The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. Compositions or compounds may also be administered as a bolus, electuary, or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms useful for oral administration include pharmaceutically acceptable emulsions, lyophiles for reconstitution, microemulsions, solutions, suspensions, syrups and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions for rectal, vaginal, or urethral administration may be presented as a suppository, which may be prepared by mixing one or more active compounds with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active compound.

Formulations of the pharmaceutical compositions for administration to the mouth may be presented as a mouthwash, or an oral spray, or an oral ointment.

Alternatively or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams or spray formulations containing such carriers as are known in the art to be appropriate.

Dosage forms for the topical or transdermal administration include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to an active compound, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention. Exemplary ophthalmic formulations are described in U.S. Publication Nos. 2005/0080056, 2005/0059744, 2005/0031697, and 2005/004074; and U.S. Pat. No. 6,583,124, the contents of which are incorporated herein by reference. If desired, liquid ophthalmic formulations have properties similar to that of lacrimal fluids, aqueous humor or vitreous humor or are compatible with such fluids.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to about 99.5% (more preferably, about 0.5 to about 90.0%) of active ingredient in combination with a pharmaceutically acceptable carrier.

In some embodiments of the invention, a compound of the invention is conjointly administered with one or more additional compounds/agents. In certain such embodiments, the one or more additional agents are selected from PPAR agonists and antagonists, AMPK activators, PARP inhibitors, SIRT-activating compounds, and acetyl-CoA carboxylase inhibitors, and the pharmaceutically acceptable salts of these compounds.

In certain such embodiments, the conjoint administration is simultaneous. In certain such embodiments, the compound of the invention is co-formulated with the one or more additional compounds. In certain other such embodiments, the compound of the invention is administered separately but simultaneously with the one or more additional compounds. In certain such embodiments, the conjoint administration is sequential, with administration of the compound of the invention preceding or following the administration of the one or more additional compound by minutes or hours.

Methods of introduction of a compound of the invention may also be provided by rechargeable or biodegradable devices. Various slow release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed, or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated, and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. “Therapeutically effective amount” refers to the concentration of a compound that is sufficient to elicit the desired therapeutic effect. It is generally understood that the effective amount of the compound will vary according to the weight, sex, age, and medical history of the subject. Other factors that influence the effective amount may include, but are not limited to, the severity of the patient's condition, the disorder being treated, the stability of the compound, and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six, or more sub-doses administered separately at appropriate intervals throughout the day, optionally, in unit dosage forms. In certain embodiments of the present invention, the active compound may be administered two or three times daily. In preferred embodiments, the active compound will be administered once daily.

The patient receiving this treatment is any animal in need, including primates, in particular humans, and other mammals such as equines, cattle, swine and sheep; and poultry and pets in general.

In certain embodiments, compounds of the invention may be used alone or conjointly administered with another type of therapeutic agent. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a separate formulation, either concomitantly or sequentially. In certain embodiments, the different therapeutic compounds can be administered within one hour, 12 hours, 24 hours, 36 hours, 48 hours, 72 hours, a week, or more of one another. Thus, an individual who receives such treatment can benefit from a combined effect of different therapeutic compounds.

This invention includes the use of pharmaceutically acceptable salts of compounds of the invention in the compositions and methods of the present invention. In certain embodiments, contemplated salts of the invention include, but are not limited to, alkyl, dialkyl, trialkyl, or tetra-alkyl ammonium salts. In certain embodiments, contemplated salts of the invention include, but are not limited to, L-arginine, benenthamine, benzathine, betaine, calcium hydroxide, choline, deanol, diethanolamine, diethylamine, 2-(diethylamino)ethanol, ethanolamine, ethylenediamine, N-methylglucamine, hydrabamine, 1H-imidazole, lithium, L-lysine, magnesium, 4-(2-hydroxyethyl)morpholine, piperazine, potassium, 1-(2-hydroxyethyl)pyrrolidine, sodium, triethanolamine, tromethamine, and zinc salts. In certain embodiments, contemplated salts of the invention include, but are not limited to, Na, Ca, K, Mg, Zn, or other metal salts.

The pharmaceutically acceptable acid addition salts can also exist as various solvates, such as with water, methanol, ethanol, dimethylformamide, and the like. Mixtures of such solvates can also be prepared. The source of such solvate can be from the solvent of crystallization, inherent in the solvent of preparation or crystallization, or adventitious to such solvent.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol, and the like; and (3) metal-chelating agents, such as citric acid, ethylenediamine tetraacetic acid (EDTA), sorbitol, tartaric acid, phosphoric acid, and the like.

The invention now being generally described, it will be more readily understood by reference to the following examples, which are included merely for purposes of illustration of certain aspects and embodiments of the present invention, and are not intended to limit the invention.

### EXAMPLES

### Example 1

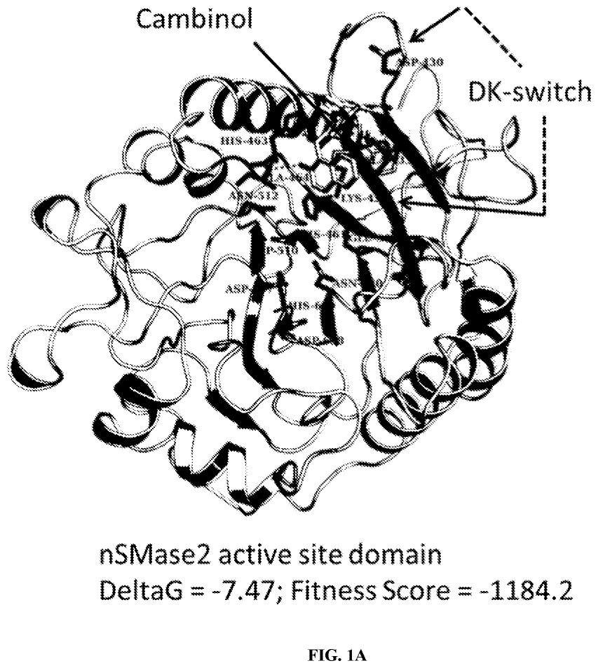

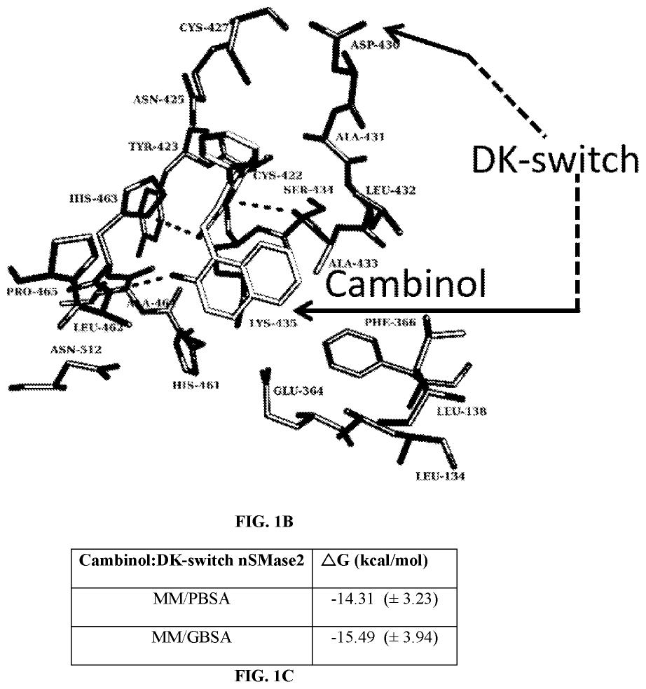

### Docking and Binding Calculations for Cambinol and nSMase2 (FIG. 1)

Cambinol is shown herein to be an a inhibitor binding to the active site domain of n-SMase2, on the basis of virtual screening to be able to prevent the spread of Tau seeds from “donor” cells to “recipient” cells. Molecular docking studies with cambinol and the n-SMase2 catalytic domain (using PDB id: 5UVG) were conducted and this displayed that cambinol can bind at the active site of n-SMase2 in the area of the DK-switch and lock the switch in the open conformation, the DK-switch is represented by residues Asp-430 (D) and Lys-435 (K), causing the enzyme to be in the inactive or inhibited state. The predicted deltaG of the n-SMase2-cambinol binding is −7.47, with a calculated fitness score of −1184.2.

The simulation calculation of cambinol binding to the DK-switch using the MM/PBSA and MM/GBSA modules in Amber and it shows that cambinol binds to the DK-switch region over the entire time of the simulation study.

### Example 2

### Synthesis of Exemplary Compounds

A exemplary synthetic scheme for cambinol and analogs thereof is shown below.

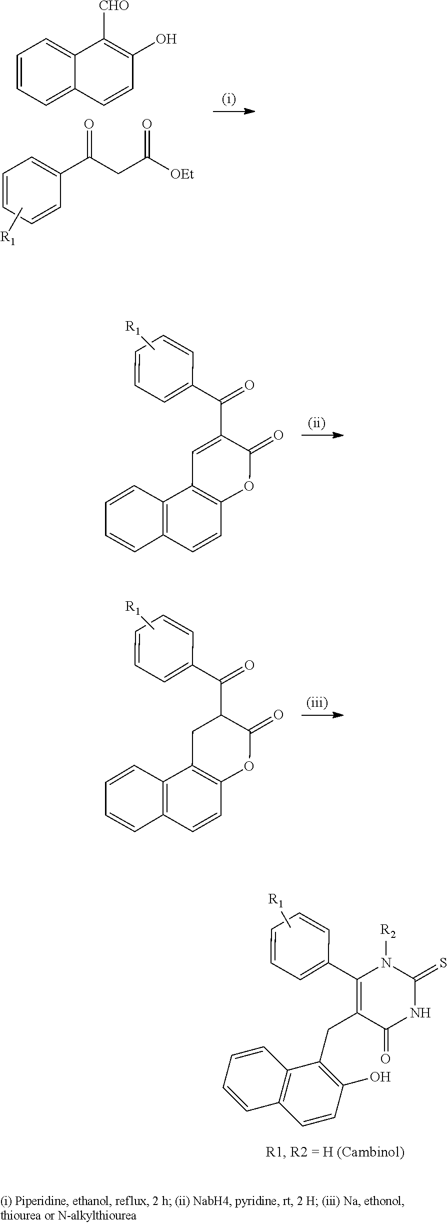

The synthesis of related compounds is described in Federico Medda et al., J. Med. Chem. (2009) 52, 2673-2682.

### Example 3

### Dose Response Inhibition by Cambinol of n-SMase2

nSMase2 activity was measured in the homogenates of HEK293t cells stably and constitutively expressing human nSMase2. Cell homogenates were prepared in [100 mM Tris-HCl pH 7.5, 1 mM EDTA, 100 mM sucrose, 100 μM PMSF, 1× protease inhibitor cocktail III], then sonicated 3× for 30 sec to achieve cell lysis. The homogenates were diluted sixteen times in the reaction buffer [0.5 M Tris-HCl, 50 mM MgCl2, pH 7.4] and added into the 384 well plate (Corning #3820; 5 μl per well). Tested compounds were diluted in the reaction buffer (four times from final concentration) and added to their corresponding wells (5 μl per well) starting from 10 μM, and serially diluted to 78 nM. The reaction was started by adding 10 μL of the working solution (100 μM Amplex® Red reagent containing 2 U/mL HRP, 0.2 U/mL choline oxidase, 8 U/mL of alkaline phosphatase, and 0.5 mM sphingomyelin) and the progression of the reaction was monitored at 540/590 nm (Ex/Em) for 2 h at 37° C. The activity of nSMase2 at 2 hrs was plotted as percentage of control using PrismGraphPad. Dose-response curve for nSMase2 inhibition by cambinol is depicted in FIG. 2.

### Example 4

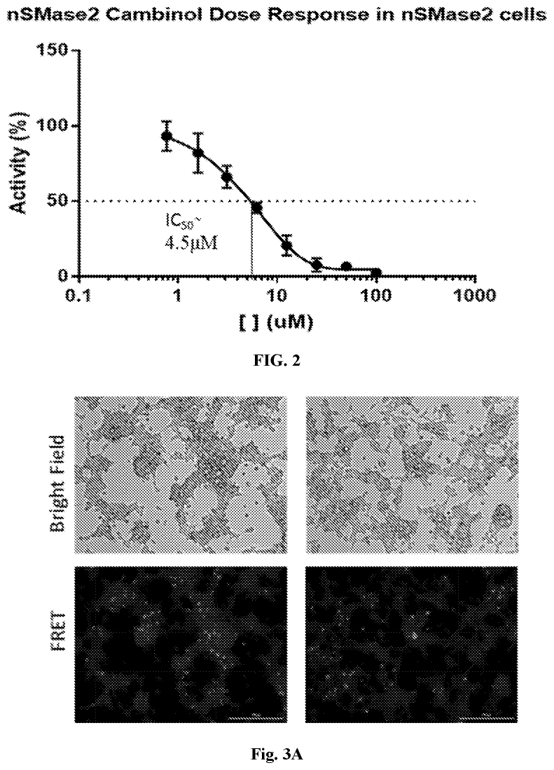

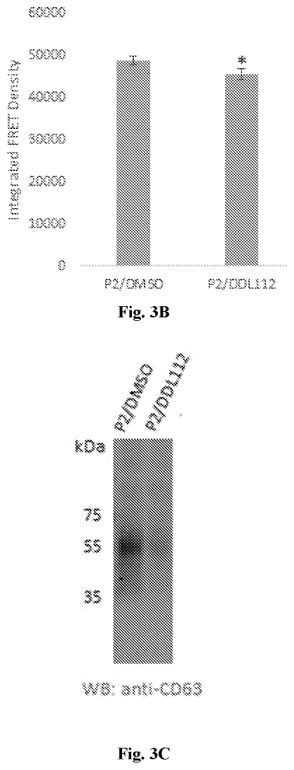

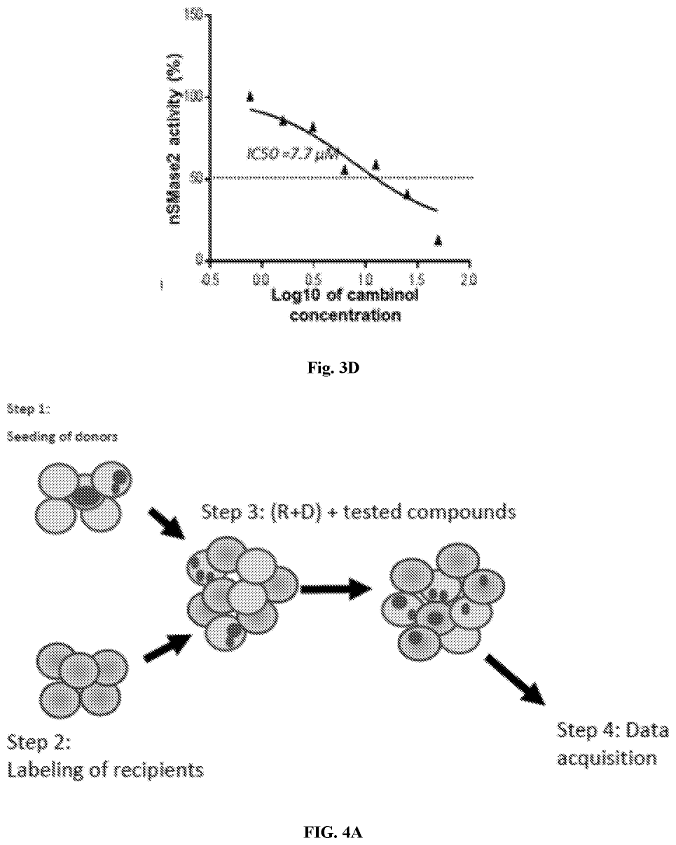

### Inhibition of P2-Induced Tau Aggregation in Tau P301S FRET Biosensors by nSMase2 Inhibitors (FIG. 3)

The tau biosensors were HEK293T cells, with stably expressed tau repeat domains with disease-associated mutation (P301S) fused to either CFP or YFP (HEK293T Tau RD P301S FRET biosensors) were transduced with pulled synaptosomal (P2) extracts from AD brains using lipofectamine 2000. Cultures treated with equal amount of empty liposomes were used as a control. Cells were incubated with liposomes for 20 hrs and then trypsinized/washed to eliminate exogenous tau seeds and re-plated in exosome-free medium (30,000 cells per well of 96-well plate) with either 10 μM of cambinol or DMSO (0.001%) for control. Cells were imaged after 24 hrs using automated microscope imaging system (Lionheart FX) equipped with 20× objective and CFP-YFP FRET Image filter cube. Treatment with the nSMase2 inhibitor visibly decreased amount of FRET-positive tau aggregates comparing to DMSO-treated control (FIG. 3A).

After sixty hours treatment, cells and cell medium were collected. Cells were prepared for flow cytometry and FRET signal was detected using LSRII flow cytometer (BD biosciences). Cells were back gated onto forward scatter versus side scatter to insure a single cell analysis, 5,000 cells were collected within the gate. FRET negative and FRET-positive cell populations were defined as previously described. Flow cytometry data presented in FIG. 3B confirmed our microscopy data described above and demonstrated significant decrease in FRET-positive signal (integrated FRET density=% of FRET pos cells×Median of FRET intensity) for inhibition of nSMase2 activity in cells with 10 uM of cambinol.

Extracellular vesicles (EVs) were purified from the cell culture medium using Exo-Quick-TC exosome purification kit (SBI, EXOTC10A-1) according to the manufacturing instructions. Immunoblot analysis of the EV fractions was done by 10-20% Tris-Glycine gel in non-reduced conditions, transferred to PVDF membrane and probed with antibodies against CD63 (ThermoFisher, 10628D), followed by HRP-conjugated secondary antibodies. Chemiluminescent signals were obtained with Super Signal West Femto substrate (Thermo Scientific Pierce 34,095) and detected using a BioSpectrum 600 imaging system and quantified using VisionWorks Version 6.6A software (UVP; Upland, Calif.). Decrease in levels of exosomal marker CD63 in cell medium from cambinol treated cells (FIG. 3C) suggests that decrease in FRET-signal in tau biosensor cells in 24 hours and 60 hours of cambinol treatment (FIGS. 3A and B) depends on EV-mediated propagation of tau seeds between the cells. Inhibition of nSMase2 activity in cell extracts of confluent tau biosensors was confirmed by nSMase2 activity assay analogous to the one described in Example 2. Calculated IC₅₀ was approximately 7.7 uM (FIG. 3D)

### Example 5

### Functional Assay for Tau Seed Transmission from Donor Cells (D) to Recipient Cells

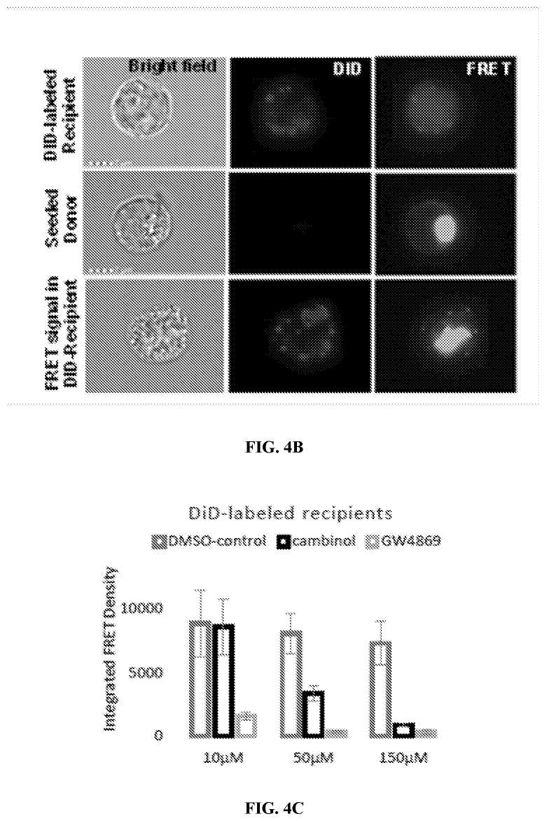

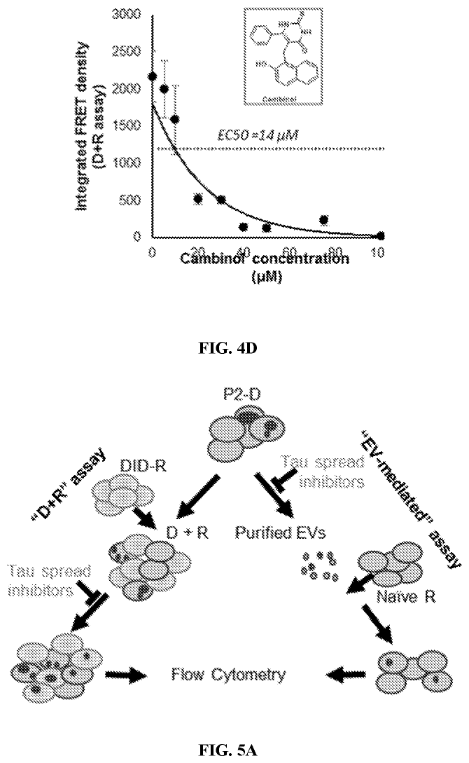

The compounds disclosed herein were further evaluated in the functional assays (“D+R approach”). The assays were based on the use of two cell populations—donor cells (D) and recipient cells (R). Recipients express a specific signal upon accumulation of protein aggregates (for example, FRET signal). The D+R approach can be divided into four steps (FIG. 4A).

In step 1, donor and recipient cells were plated in separation and protein aggregation is induced in the donor cells by using tissue-derived or synthetic proteopathic seeds (“seeding”) or by chemical and/or genetic methods. In step 2, either donor cells or recipient cells were labeled with a lipophilic dye (or by other cell labeling technology) in order to be able to distinguish between donor and recipients cells after trypsinization and re-plating of the donor and recipient cells together in step 3 (R+D). Alternatively, donor and recipient cells were physically divided by a permeable support (for example, Transwell). In any scenario, the cells were connected through the media and they grow for 24-60 hours with or without addition of pre-selected compounds. Efficiency of proteopathic seed transmission from donor cells to recipient cells can be analyzed using microscopy, flow cytometry, or imaging flow cytometry. Tau biosensor cells were adapted as a tool for the implementation of the D+R approach for the evaluation of cambinol and GW4869 (n-SMase2 inhibitor, structurally and mechanistically unrelated to cambinol) effects on tau seed transmission. Treatment of the tau biosensor cells with tau seeds lead to RD aggregation, as a result, CFP and YFP molecules come into close proximity and a FRET signal can be generated in the CFP/YFP pair upon illumination. The far-red fluorescent, lipophilic carbocyanine dye, DiD, was used for labeling recipient cells. This dye allowed for distinguishing between the DiD-negative donor and the DiD positive recipient cells without interference with the CFP/YFP FRET signal. The representative images of DiD-negative FRETpositive donor cell seeded with P2-derived tau seeds), DiD-positive FRET-negative recipient cell (DiD-R), and DiD positive FRET-positive recipient cell, which were growing in the presence of P2-D (DiD-R/R+D) are presented at the FIG. 4B. Both tested N-Smase2 inhibitors, cambinol and GW4869, restrained tau seed transmission from the donor cells to the recipient cells in a dose-dependent manner (FIG. 4C). Dose-dependent reduction in the tau transfer from donor to recipient cells after treatment with cambinol (D+R assay) with an EC₅₀ of approximately 14 uM (FIG. 4D). This data demonstrates the efficiency of the D+R approach for lead identification and optimization prior to preclinical study.

### Example 6

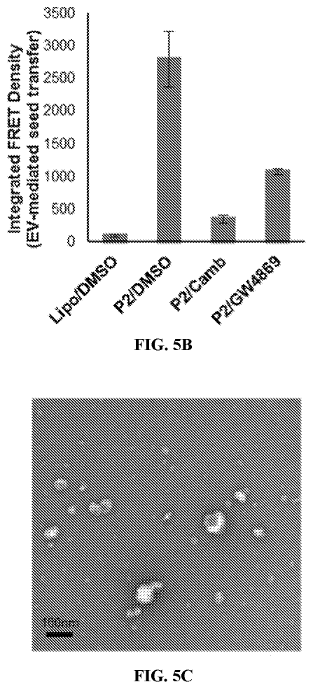

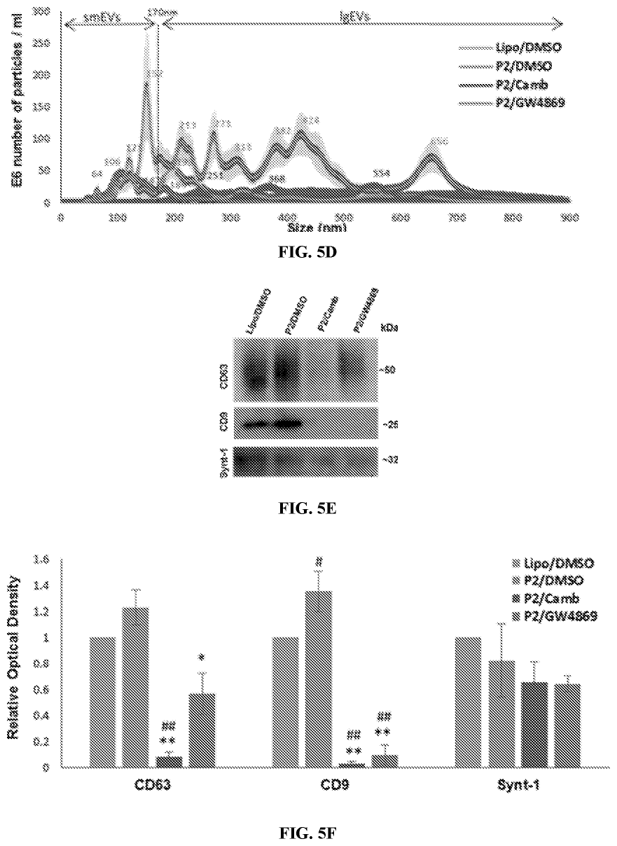

### Functional Assay for EV-Mediated Transmission of Tau Seeds (FIG. 5)

For EV (extracellular vesicles)-mediated tau transfer assay, donor cells, transduced with AD-brain derived synaptosomal extracts (P2) or synthetic K18 seeds, were cultured for 48 hours in exosome-free medium with or without indicated concentration of tested compounds or DMSO for control, then medium was collected and EVs were purified using Exo-Quick-TC exosome purification kit (SBI, EXOTC10A-1) according to the manufacturing instructions. Naive recipient cells were plated at density 30,000 cells per well (96-well plates), grown for 12 hour in regular medium and then medium was exchanged to exosome-depleted medium, contained exosomes purified from donor cells (each well of recipients received amount of exosomes purified from one well of donor cells). Recipient cells were cultured in the presence of donor cell exosomes for 60 hrs. EV-mediated transfer assay scheme is presented at the FIG. 5A (right).

Treatment of P2-seeded donor cells with cambinol (P2/Camb) or GW4869 (P2/GW4869) at 50 μM shows decrease in EV-mediated transfer of tau to recipient cells (FIG. 5B). Our analysis confirmed that nSMase2 inhibitors suppress release of EVs from P2-seeded tau biosensor cells. Four groups of samples were analyzed: Lipo/DMSO samples contained EVs released from non-seeded (empty liposome treated) cells after 48 hours of vehicle (DMSO) treatment, P2/DMSO, P2/Camb, and P2/GW4869—EVs released from P2-seeded tau biosensor cells after 48 hours of treatment with DMSO, cambinol (50 μM) or GW4869 (50 μM) treatment respectively. EV samples purified from the same number of donor cells were analyzed per each group. Purified EVs were imaged by transmission electron microscopy (FIG. 5C). Size distribution of particles in the samples were assessed by nanoparticle tracking analysis (FIG. 5D). WB analysis for exosome markers also confirmed decrease in the exosome-specific marker expression (FIG. 5E). Densitometry analysis from WB is presented in FIG. 5F.

### Example 7

### Pharmacokinetic and Target Engagement Studies

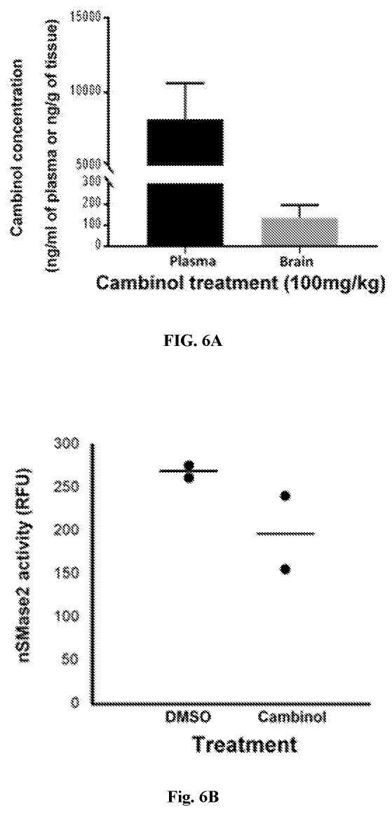

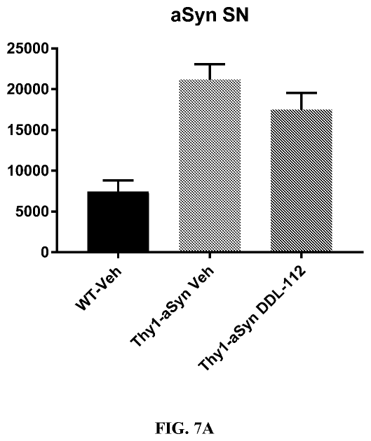

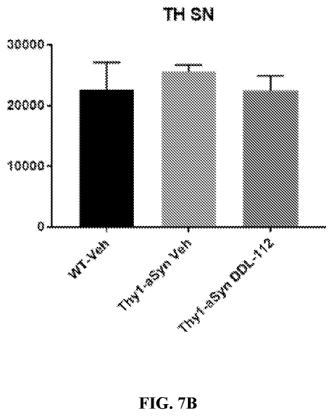

In vivo pharmacokinetic (PK) and target-engagement (nSMase 2 inhibition) study showed that cambinol (DDL-112) has low brain/plasma ratio, in a good agreement with the StarDrop analysis (predicted BBB penetration −1.154). C57BL6 mice were treated either with 100 mg/kg of cambinol or corresponding amount of vehicle (DMSO) using oral gavage and sacrificed 4 hours later (2 mice per group). At that time point only 135.3±60.9 ng/g of cambinol was detected in the brain by LC-MS/MS analysis, while 8150±2450 ng/ml was detected in plasma, showing a low brain/plasma ratio (FIG. 6A). However, even these low levels of DDL-112 show some inhibition of nSMase activity by 26.8% in cortical homogenates from mice treated with DDL-112 as compared to vehicle (FIG. 6B).

### Example 8

### Initial Efficacy Testing

Mice overexpressing human wild-type a-synuclein under control of the Thy-1 promoter (Thy1-aSyn mice) and control C57B1/6 mice were housed under a 12-hour light/dark cycle and had access to standard chow ad libitum. Four-month old mice (n=9 per group) we treated with cambinol (100 mg/kg/day) or corresponded amount of vehicle (10% DMSO/90% PEG) daily for 5 weeks using oral gavage. Two hours after the last dose, mice were euthanized by ketamine/xylazine over-anesthesia followed by cardiac puncture and saline transcardial perfusion. Brain tissue from individual mice were dissected.

### Immunofluorescence Staining and Quantification.

All staining procedures and data collection were performed by investigators blinded to treatment. Double-immunofluorescence for Clone 42 Anti-aSyn/TH in SN (substantia nigra). Analysis of a-synuclein and tyrosine hydroxylase (TH, the marker of dopaminergic neurons) levels was performed in serially sectioned, free-floating vibratome sections. Brain sections (SN at Bregma −3.52 mm) were washed in 50 mM TBS (pH 7.6) and incubated in mouse IgG blocking reagent (M.O.M., Vector Laboratories; Burlingame, Calif.) for 1 hour. After a quick wash in TBS for 5 minutes, sections were incubated in 10% normal goat serum (NGS)/0.5% Triton X-100/TBS for 1 hour. Sections were then incubated in mouse anti-aSyn (1:250, BD Biosciences Clone 42) and rabbit anti-TH antibody (1:1,000, Pel-Freeze) in 5% NGS at 4° C. overnight. After washing, sections were incubated in Alexa 647 goat anti-mouse IgG (1:500; Invitrogen; Carlsbad, Calif.) and Cy3 goat anti-rabbit IgG (1:500; Jackson Immunoresearch; West Grove, Pa.) in 5% NGS for 2 hours at room temperature. After washing, sections were mounted in tap water on plain glass slides.

### Quantification of Fluorescence Intensity in SN

For the quantification of aSyn and TH fluorescent immunolabeling images of immunofluorescence-labeled sections were acquired using an Agilent (Santa Clara, Calif.) microarray scanner equipped with a krypton/argon laser (647 nm) at 10 μm resolution with the photomultiplier tube set at 5% for aSyn and 10% for TH. Immunofluorescence intensity was measured using ImageJ in the appropriate region of interest. TH staining was quantified in the SN pars compact and SN pars reticulate.

### Methods

Modeling of Cambinol Interactions with nSmase2.

Molecular docking of cambinol to nSmase2 (pdbid 5UVG) was performed using the Swiss Dock server. Prior to docking, missing regions in the nSmase2 crystal structure were built using the MODELLER program. All rotatable single bonds were allowed to rotate in cambinol and the docking results were screened and analyzed with the Chimera program. MD simulation was carried out to determine the binding free energy of cambinol binding to nSmase2. AMBER16 package was used to perform the MD simulation . Antechamber module in AMBER was used to generate the parameters for cambinol. After adding hydrogens, the nSmase2-cambinol complex was solvated in a truncated octahedral TIP3P box of 12 Å, and the system was neutralized with sodium ions. Periodic boundary conditions, Particle Mesh Ewald summation and SHAKE-enabled 2-femto seconds time steps were used. Langevin dynamics temperature control was employed with a collision rate equal to 1.0 ps₋₁. A cutoff of 13 Å was used for nonbonding interactions. Initial configurations were subjected to a 1000-step minimization with the harmonic constraints of 10 kcal·mol₋₁·A°₋₂ on the protein heavy atoms. The system was gradually heated from 0 to 300 K over a period of 50 ps with harmonic constraints. The simulation at 300° K was then continued for 50 ps during which the harmonic constraints were gradually lifted. The system was then equilibrated for a period of 500 ps before the 50 ns production run. The MD simulation was carried out in the NPT ensemble. Equilibration and production run were carried out using the Sander and PMEMD modules (optimized for CUDA) of AMBER 16.0 (ff14SB), respectively. All analyses were performed using AmberTools 16 (module cpptraj). From the 50 ns simulation 10000 structures were taken at an interval 5 ps for the free energy calculations. The binding free energy for cambinol to nSMase2 were estimated using the MM/PBSA and MM/GBSA modules in AMBER by taking snapshots (10000) at every 5 pico seconds from the 50 ns production run as shown in FIG. 1.

nSMase2 Activity Assay

nSMase2 activity measured in the homogenates of HEK293T cells stably and constitutively expressing human nSMase2, homogenates of tau biosensor cells (HEK293T Tau RD P301S FRET biosensors) and in brain homogenates. Cell or tissue homogenates were prepared in [100 mM Tris-HCl pH 7.5, 1 mM EDTA, 100 mM sucrose, 100 μM PMSF, 1× protease inhibitor cocktail III], then sonicated 3× for 30 sec to achieve cell lysis. The homogenates were diluted sixteen times in the reaction buffer [0.5 M Tris-HCl, 50 mM MgCl2, pH 7.4] and added into the 384 well plate (Corning #3820; 5 μl per well). Tested compounds were diluted in the reaction buffer (four times from final concentration) and added to their corresponding wells (5 μl per well) starting from 10 μM, and serially diluted to 78 nM.

Alternatively, mice were treated with the compounds by a parental route and after euthanization the brain is removed and homogenized using 100 mM Tris-HCl pH 7.5, 100 mM sucrose, 100 μM PMSF, 1× protease inhibitor cocktail, and diluted to 2× in reaction buffer prior to the assay and nSMase2 activity is measured. The reaction was started by adding 10 μL of the working solution (100 μM Amplex Red reagent containing 2 U/mL HRP, 0.2 U/mL choline oxidase, 8 U/mL of alkaline phosphatase, and 0.5 mM sphingomyelin) and the progression of the reaction was monitored at 540/590 nm (Ex/Em) for 2 h at 37° C. The activity of nSMase2 at 2 hrs was plotted as percentage of control using PrismGraphPad.

### Preparation of Human Brain Derived Synaptosomes (P2)

Brain autopsy samples were obtained from the University of California Irvine and University of Southern California AD Research Centers. Brain tissue was cryopreserved and synaptosomal fractions (P2-fractions, or P2) were prepared as previously described . In order to prepare P2-extracts, aliquots were quickly defrosted at 37° C. and centrifuged at 10,000 g for 10 minutes at 4° C. to remove P2 from sucrose. After aspirating the supernatant, cold PBS was added to each sample in a 1:5 weight/volume ratio. Samples were then sonicated in 10-second intervals three times, incubated on ice for 30 minutes and centrifuged at 20,000 g for 20 minutes at 4° C. P2 extracts were collected and stored at −80° C.

### Functional Assays for In Vitro Screening of Tau Propagation Inhibitors.

HEK293T Tau RD P301S FRET biosensor cells were growing in the Dulbecco's Modified Eagle's medium (DMEM) with higher glucose, 10% FBS, and 1% Penicillin-streptomycin at 37° C./5% CO₂. Cells were plated in 10-sm dishes (3 million cells per dish in the regular medium) and grown for 12 hrs. Cells assigned to be donors were transduced with pulled synaptosomal (P2) extracts from AD cases using lipofectamine 2000, according to the published literature with minor modifications. We used 35 μg of pulled AD or control (NL) material per one 10-sm dish (TableS1). Defrosted P2 extracts were sonicated in water bath sonicator for 10 minutes, and diluted with Opti-MEM serum reduced medium to the final volume 200 μL per 10-sm dish. In a separate tube lipofectamine 2000 were combined with Opti-MEM medium, based on 25 μl of lipofectamine and 175 μL OptiMEM medium per each 10-sm dish, and incubated for 10 minutes at room temperature (RT). Each P2 or NL extract mix (200 μl) and prepared lipofectamine 2000 mix (200 μl) were combined and incubated for 20 min at RT. Each 10-sm dish with cell received 400 μl of the final liposomes in the total volume of medium 5 ml per dish. Cultures treated with equal amount of empty liposomes were used as an additional control group. Cells were incubated with liposomes for 20 hrs.

For “D+R” assay recipient cells were labeled with 1,1′-Dioctadecyl-3,3,3′,3′-tetramethylindodicarbocyanine (DiD) according to manufacturer's protocol (ThermoFisher V22887) on day 2. On day 3, donor cells were trypsinized (0.05% Trypsin/EDTA), washed with fresh exosome-free medium contained 10% of exosome-depleted FBS (ThermoFisher, A2720803). DiD-labeled recipient cells were also trypsinized and washed in exosome-free medium. For “D+R” assay, donor cells were cultured with DiD-labeled recipient cells in 1:1 ratio (15,000 donor cells and 15,000 recipient cells per well of 96-well plate) for 48 hours for with or without indicated concentration of tested compounds or dimethyl sulfoxide (DMSO) for control. For EV (extracellular vesicles)-mediated tau transfer assay, donor cells were cultured for 48 hours in exosome-free medium (35,000 donor cells per well of 96-well plate) with or without indicated concentration of tested compounds or DMSO for control, then medium was collected and EVs were purified using Exo-Quick-TC exosome purification kit (SBI, EXOTC10A-1) according to the manufacturing instructions. Naive recipient cells were plated at density 30,000 cells per well (96-well plates), grown for 12 hour in regular medium and then medium was exchanged to exosome-depleted medium, contained exosomes purified from donor cells (each well of recipients received amount of exosomes purified from one well of donor cells). Recipient cells were cultured in the presence of donor cell exosomes for 60 hrs. Recipients and donor cells (D+R assay) or recipients only cells (EV-mediated tau transfer assay) were collected and prepared for flow cytometry as previously described. FRET signal was detected using LSRII flow cytometer (BD biosciences). Cell were back-gated onto forward scatter versus side scatter to insure a single cell analysis, 5,000 cells were collected within the gate. FRET negative and FRET-positive cell populations were defined as previously described in the literature. For imaging, cells were prepared as above, but cells from six wells (96-well plate) were combined per each condition and diluted in 25 μL of flow cytometry buffer. The ImageStream Mark II imaging flow cytometry system was used for data acquisition. FRET (CFP/YFP) signal was excited by 405 nm laser for CFP excitation and detected in the YFP image detection channel. Single cells were gated based on the bright field, and 10,000 images (60×) were collected within the gate. Data were analyzed using the IDEAS data analysis software. Cell viability was assessed with LDH assay (Promega, G1780).

### EV Purification and Characterization

Donor cells were grown in medium with exosome-depleted FBS (ThermoFisher, A2720803) for 48 hours with or without compounds, cell culture medium was collected and EVs purified using ExoQuick-TC kit (SBI biosciences). For quality control, small amounts of purified EVs were fixed on a copper mesh in glutaraldehyde/paraformaldehyde solution, stained with 2% uranyl acetate solution and imaged on a JEOL 100CX electron microscope at 29,000 times magnification. The remaining samples were either sent for nanoparticle tracking analysis (NTA) at Alpha Nanotech or used for biochemical characterization. Immunoblot analysis was done by 10-20% Tris-Glycine gel in non-reduced conditions, transferred to PVDF membrane and probed with antibodies against CD63 (ThermoFisher, 10628D), CD9 (ThermoFisher, 10626D), and syntenin-1 (sc-48742), followed by HRP-conjugated secondary antibodies. Chemiluminescent signals were obtained with Super Signal West Femto substrate (Thermo Scientific Pierce 34095) and detected using a BioSpectrum 600 imaging system and quantified using VisionWorks Version 6.6A software (UVP; Upland, Calif.).

In Vivo Treatment with Cambinol

Brain permeability and target engagement studies were conducted in C57BL/6J mice. Cambinol stock solution (60 mg/ml) was prepared in DMSO. Four hours after oral gavage treatment with cambinol as a single 100 mg/kg dose, or vehicle (2 mice per group), mice were sacrificed and tissue samples (brain and plasma) collected. Drug level evaluation in brain and plasma samples was performed by LC-MS/MS analysis. Brain tissue was homogenized and nSMase2 activity measured as described above.

### Cambinol Study in Parkinson's Disease (PD) Mice.

Mice overexpressing human wild-type a-synuclein under control of the Thy-1 promoter (Thy1-aSyn mice) and control C57B¹/₆ mice were housed under a 12-hour light/dark cycle and had access to standard chow ad libitum. Four-month old mice (n=9 per group) we treated with cambinol (100 mg/kg/day) or corresponded amount of vehicle (10% DMSO/90% PEG) daily for 5 weeks using oral gavage. Two hours after the last dose, mice were euthanized by ketamine/xylazine over-anesthesia followed by cardiac puncture and saline transcardial perfusion. Brain tissue from individual mice were dissected.

### Immunofluorescence Staining and Quantification.

All staining procedures and data collection were performed by investigators blinded to treatment.

Double-Immunofluorescence for Clone 42 Anti-aSyn/TH in SN (Substantia nigra).

Analysis of α-synuclein and tyrosine hydroxylase (TH, the marker of dopaminergic neurons) levels was performed in serially sectioned, free-floating vibratome sections. Brain sections (SN at Bregma −3.52 mm) were washed in 50 mM TBS (pH 7.6) and incubated in mouse IgG blocking reagent (M.O.M., Vector Laboratories; Burlingame, Calif.) for 1 hour. After a quick wash in TBS for 5 minutes, sections were incubated in 10% normal goat serum (NGS)/0.5% Triton X-100/TBS for 1 hour. Sections were then incubated in mouse anti-aSyn (1:250, BD Biosciences Clone 42) and rabbit anti-TH antibody (1:1,000, Pel-Freeze) in 5% NGS at 4° C. overnight. After washing, sections were incubated in Alexa 647 goat anti-mouse IgG (1:500; Invitrogen; Carlsbad, Calif.) and Cy3 goat anti-rabbit IgG (1:500; Jackson Immunoresearch; West Grove, Pa.) in 5% NGS for 2 hours at room temperature. After washing, sections were mounted in tap water on plain glass slides.

### Quantification of Fluorescence Intensity in SN.

For the quantification of aSyn and TH fluorescent immunolabeling images of immunofluorescence-labeled sections were acquired using an Agilent (Santa Clara, Calif.) microarray scanner equipped with a krypton/argon laser (647 nm) at 10 μm resolution with the photomultiplier tube set at 5% for aSyn and 10% for TH. Immunofluorescence intensity was measured using ImageJ in the appropriate region of interest. TH staining was quantified in the SN pars compact and SN pars reticulate.

### Statistical Snalysis

All the data was expressed as the mean±SEM. Significant differences were determined by one-way ANOVA with post hoc Tukey multiple comparisons test using online web statistical calculator (http://astatsa.com/OneWay_Anova_with_TukeyHSD). Values of *<0.05 and **<0.01 were considered statistically significant.

### Results

Cell-Free Assay for nSMase2 Activity

The dose response shows cambinol inhibition of nSMase2 in overexpressing cells with an IC50 of 4.5 μM. Similarly it was found that nSMase2 activity was elevated in confluent tau biosensor cells and in confluent SH-SYSY human neuroblastoma cells. Cambinol shows dose-dependent inhibition of nSMase2 activity in cell extracts from tau biosensor cells with an IC50 of ˜7.7 μM similar to the reported value of 5 μM.

Cambinol inhibits EV-mediated cell-to-cell tau propagation. We demonstrate that cambinol inhibits tau seed spread from donor to recipient cells in the “D+R” functional assay. Pooled synaptosomal (P2) extracts from cryopreserved human AD brains were used to seed tau aggregation in donor cells. Higher levels of seed-competent tau were found in the synaptosomal extracts, based on the donor cell seeding. For negative controls, donor cells were transduced with either non-pathological (NL) brain extracts or empty liposomes. Co-culturing of 1,1′-Dioctadecyl-3,3,3′,3′-tetramethylindodicarbocyanine (DiD)-labeled recipient cells with P2-seeded donor cells led to the transfer of tau seeds from donor to recipient cells and the appearance of FRET-positive tau inclusions in a small but detectable (˜3-5%) population of DiD-labeled recipient cells. Of note, donor cells were trypsinized and washed before co-culturing them with recipient cells to avoid their contact with human brain-derived material and liposomes, used for P2 delivery to the donor cells. Only the top 30% of DiD-positive cells, based on signal intensity, were considered recipients to overcome potential DiD cell-to-cell transfer interference. Imaging flow cytometry data confirmed the presence of FRET-positive aggregates in the cells labeled with DiD. This data shows that 48-hour treatment with cambinol inhibited the formation of FRET-positive tau aggregates in the recipient cells in a dose-dependent manner with EC50 of 14 μM. Neither P2-seeding nor treatment with nSMase2 inhibitors caused a significant cell death based on the LDH assay. Further, we purified EVs from donor cell media and evaluated their tau propagation potential. Our data confirmed previously reported EV-mediated transfer of tau seeds from tau biosensor cells to naive recipient cells and demonstrated that inhibition of nSMase2 in donor cells by either cambinol or GW4869, significantly reduces this selective EV-mediated transfer by 8 and 2.6 times respectively.

### Characterization of Cell-Derived EVs.

Transmission electron microscopy (TEM) and nanoparticle tracking analysis (NTA) were employed to evaluate morphology, size, and number of the extracellular particles released by donor cells. The presence of classic “cup”-shaped vesicles of different sizes in the EV preparations was confirmed by TEM. NTA revealed 3.5 times more particles released from P2-seeded donors (P2/DMSO sample) compared to non-seeded control cells (Lipo/DMSO sample). Seeding with P2 extracts also led to the shift in the size distribution of smaller particles (<170 nm), at size 153 nm in P2/DMSO compared to 125 nm in the LIPO/DMSO sample, and the appearance of additional larger particle size range (>170 nm). Donor cells which were treated with cambinol (P2/Camb) or GW4869 (P2/GW4869) released less EVs, as expected. Interestingly, nSMase2 inhibitors not only reduced the number of small “exosome-like” particles but also inhibited the formation of larger particles. The nature of the larger particles is not clear; they could represent populations of ectosomes, or aggregates of smaller EVs. These larger particles are unlikely to be cell fragments, apoptotic bodies, or passively release aggregates, given previously reported low toxicity of AD derived tau strain and the lack of any significant cell toxicity of nSMase2 inhibitors based on the LDH assay. Biochemical characterization of EV fractions for known exosome-enriched markers—CD63, CD9, and syntenin-1, by immunoblot was performed. Levels of CD9, but not CD63 or syntenin-1, was significantly elevated in P2/DMSO samples as compared to the Lipo/DMSO control. Levels of tetraspanins, but not syntenin-1, were significantly decreased in EV samples from nSMase2 inhibitor treatment.

Brain Permeability and Target Engagement Study with Cambinol.

After oral administration of cambinol (100 mg/kg dose) levels of 135.3±60.9 ng/g were detected in the brain at 4 h, while levels in plasma was 8150±2450 ng/ml, showing that cambinol has a low brain/plasma ratio. Furthermore, at 4 hours after cambinol treatment we observe a decrease in nSMase activity in brain homogenates (by ˜26.8%) in treated mice compared to controls, thus indicating potential in vivo target engagement.

Modeling of Cambinol Binding to nSMase2 and Interactions.

The SwissDock web server was used to predict the cambinol binding to nSMase2. The free energy value indicates that cambinol binding to nSMase2 at the DK-switch is thermodynamically favorable. Cumulatively, the MD simulation data suggest that cambinol is inhibiting nSMase2 activity by preventing the conformational transition process at the DK-switch region and the interaction between Asp430 and Lys435 at the active site domain of nSMase2.

### Discussion

Elucidation of molecular pathways that are involved in cell-to-cell propagation of pathological tau species could provide novel avenues for therapeutic development to suppress tau pathology propagation and attenuate cognitive decline in Alzheimer's disease and other tauopathies. Herein we report on the identification of a screening hit cambinol by employing tau biosensor cells. This example provides evidence that cambinol, a nSMase2 inhibitor, can reduce tau spread from donor cells, seeded with synaptosomal (P2) fractions from AD brain samples to recipient cells; this inhibition is dose-dependent with an EC₅₀ of approximately 14 μM. Our studies demonstrate that cambinol is more effective than the nSMase2 inhibitor GW4869 in suppressing release of EVs from P2-seeded donor cells and EV-mediated transfer of tau seeds from donor to recipient cells.

Unexpectedly, P2-seeded tau biosensor cells released 3.5 times more particles compared to cells treated with empty liposomes. In our studies, human brain P2 extracts had higher potential to induce propagation compared to synthetic K18 tau seeds in D+R assay. This unexpected effect could be due to the presence of biological mediators of nSMase2, in the AD brain-derived material, such as amyloid beta, tumor necrosis factor alpha or specific tau conformers. Biochemical examination of known exosome markers CD63, CD9 and syntenin-lin the EV fractions by immunoblotting suggests that tau aggregation may interfere with some but not other exosome biogenesis pathways. Specifically, our data suggests that P2-induced tau aggregation may not have a significant effect on the syndecan-syntenin pathway. An initial in vivo study shows that cambinol can reduce the level of the nSMase2 activity in the brain after oral administration at a dose of 100 mg/kg. These data suggest that cambinol can engage the target nSMase2 in the brain and is therefore a promising molecular probe for further evaluation of this mechanism in tauopathy models.

Furthermore, for the first time, it is shown that through molecular docking and simulation studies that inhibition of nSMase2 by cambinol may involve a novel binding mechanism at the active site of nSMase2. These modeling studies reveal that cambinol can target the DK-switch of nSMase2, and thus modulate the interaction of catalytic residues D430 and K435 resulting in inhibition of the nSMase2 enzyme activity. In contrast, the cationic GW4869 was reported to bind outside the active site domain interacting with the anionic phosphatidylserine involved in activation of nSMase2.

The physiological level of nSMase2 expression is important for normal brain function. The activity of nSMase2 in the brain has been reported to increase with age, which could lead to dysregulation in sphingomyelin turnover. Moreover, brain ceramide levels were found to be elevated in AD compared to age-matched control subjects, with a higher order of ceramide/sphingomyelin imbalance in ApoE4 carriers. Thus, elevated nSMase2 activity leading to abnormal exosome release may be an important contributing factor in age-related tau pathology spread. The discovery of cambinol and its interactions with the DK-switch of nSMase2 will provide new avenues for targeted design of small molecule inhibitors of this enzyme with advanced drug-like properties. New orally active, brain permeable and efficacious nSMase2 inhibitors, such as the compounds disclosed herein, are urgently required as preclinical candidates for testing in tauopathy models.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A compound having a structure of Formula (I): 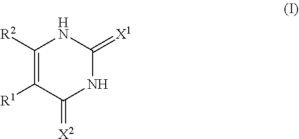
or a pharmaceutically acceptable salt thereof, wherein:
X¹ and X² are each independently O or S; and
R¹ and R² are each, independently alkyl, —C(O)-alkyl, haloalkyl, alkoxy, cycloalkyl, aryl, —C(O)-aryl, aralkyl, or haloalkyl, each of which is unsubstituted or substituted with one or more substituents;
provided that the compound of Formula (I) is not a compound of Formula (A): 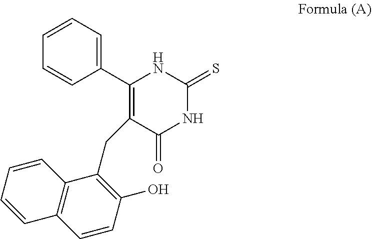 [SMILES: O=c1[nH]c(=S)[nH]c(-c2ccccc2)c1Cc1c(O)ccc2ccccc12]
or a pharmaceutically acceptable salt thereof.

2. The compound of claim 1, wherein the compound has a structure of Formula (Ia) or (Ib): 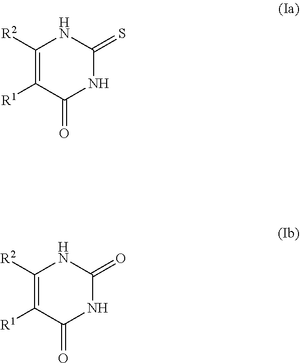
or a pharmaceutically acceptable salt thereof.

3. (canceled)

4. The compound of claim 1, wherein R¹ and R² are each, independently —C(O)—C₁-C₄-alkyl, —C₁-C₄-alkyl, aryl, —C(O)-aryl, or aralkyl, each of which is unsubstituted or substituted with one or more substituents.

5. (canceled)

6. The compound of claim 4, wherein R¹ and R² are each, independently —C(O)—C₁-C₄-alkyl, —C₁-C₄-alkyl, aryl, —C(O)-aryl, or aralkyl, each of which is substituted with halogen, OH, —C₁-C₄-alkyl, —C₁-C₄-haloalkyl, —C₁-C₄-alkoxy, or —C₁-C₄-haloalkoxy.

7. The compound of claim 1, wherein the compound is selected from: 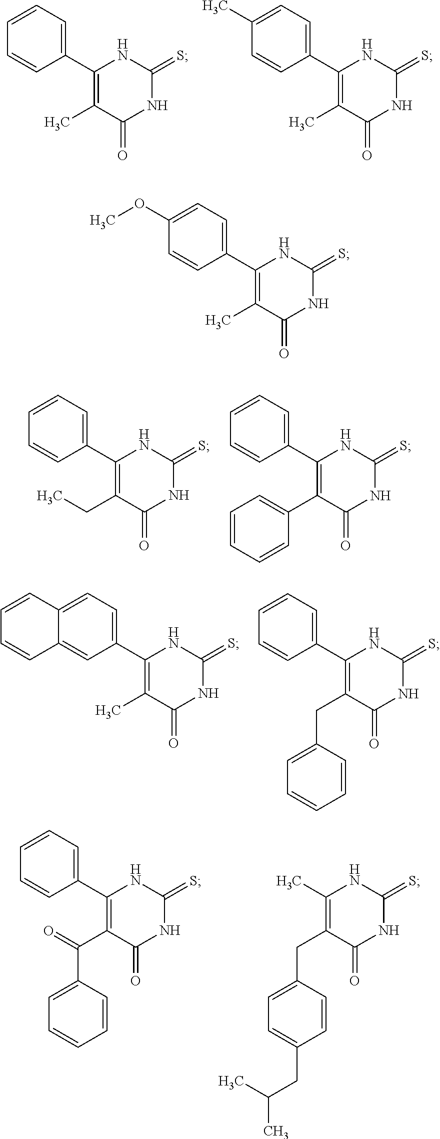 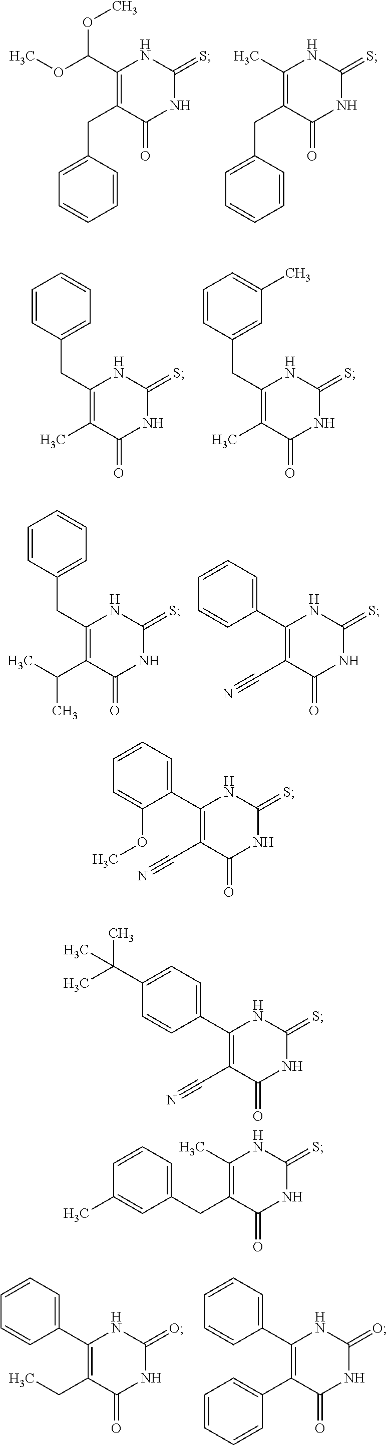 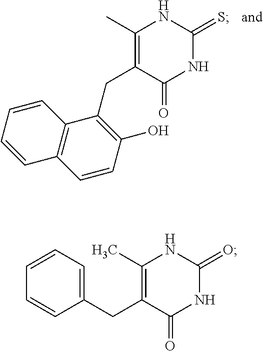 [SMILES: Cc1[nH]c(=S)[nH]c(=O)c1Cc1c(O)ccc2ccccc12 ; Cc1[nH]c(=O)[nH]c(=O)c1Cc1ccccc1]
or a pharmaceutically acceptable salt thereof.

8. A compound having a structure of Formula (II): 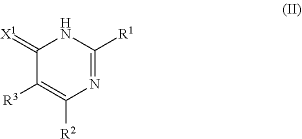
or a pharmaceutically acceptable salt thereof, wherein:
X¹ is O or S;
R¹ is alkyl, alkenyl, aryl, aralkyl, heteroaryl, —O-alkyl, —O-alkenyl, —O-haloalkyl, —O-cycloalkyl, —O-aralkyl, —O-aralkoxy, —O-aryl, 13 O-heteroaryl, —S-alkyl, —S-alkenyl, —S-haloalkyl, —S-cycloalkyl, —S-aralkyl, —S-aralkoxy, —S-aryl, or —S-heteroaryl, each of which is unsubstituted or substituted with one or more substituents; and
R² and R³ are each, independently CN, C(O)-alkyl, alkyl, haloalkyl, alkoxy, cycloalkyl, aryl, aralkyl, alkyl-O-aryl, or heteroaryl, wherein each of —C(O)-alkyl, alkyl, haloalkyl, alkoxy, cycloalkyl, aryl, aralkyl, aryl-O-alkyl, or heteroaryl is unsubstituted or substituted with one or more substituents.

9. The compound of claim 8, wherein the compound has a structure of Formula (IIa) or (IIIb): 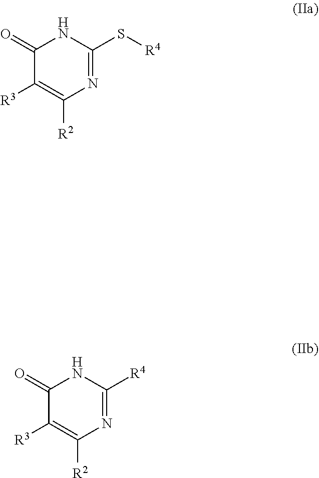
or a pharmaceutically acceptable salt thereof, wherein R⁴ is alkyl, alkenyl, haloalkyl, cycloalkyl, aralkyl, aralkoxy, aryl, or heteroaryl, each of which is unsubstituted or substituted with one or more substituents.

10. (canceled)

11. The compound of claim 8, wherein R² is —C₁-C₄-alkyl, aryl, or aralkyl, each of which is unsubstituted or substituted with one or more substituents.

12. (canceled)

13. The compound of claim 11, wherein R² is C₁-C₄-alkyl, aryl, or aralkyl, each of which is substituted with halogen, —OH, —C₁-C₄-alkyl, —C₁-C₄-haloalkyl, —C₁-C₄-alkoxy, or —C₁-C₄-haloalkoxy.

14. The compound of claim 8, wherein R³ is —CN, —C(O)—C₁-C₄-alkyl, —C₁-C₆-alkyl, aryl, or aralkyl, wherein —C(O)—C₁-C₄-alkyl, —C₁-C₆-alkyl, aryl, or aralkyl is unsubstituted or substituted with one or more substituents.

15. (canceled)

16. The compound of claim 14, wherein R³ is —C(O)—C₁-C₄-alkyl, —C₁-C₆-alkyl, aryl, or aralkyl, each of which is substituted with halogen, OH, —C₁-C₄-alkyl, —C₁-C₄-haloalkyl, —C₁-C₄-alkoxy, or —C₁-C₄-haloalkoxy.

17. The compound of claim 8, wherein R⁴ is —C₁-C₄-alkyl, —C₂-C₄-alkenyl, aryl, aralkyl, or aryl-O—C₁-C₄-alkyl, each of which is unsubstituted or substituted with one or more substituents.

18. (canceled)

19. The compound of claim 17, wherein R⁴ is —C₁-C₄-alkyl, —C₂-C₄-alkenyl, aryl, aralkyl, or aryl-O—C₁-C₄-alkyl, each of which is unsubstituted or substituted with halogen, —OH, —C₁-C₄-alkyl, —C₁-C₄-haloalkyl, —C₁-C₄-alkoxy, or —C₁-C₄-haloalkoxy.

20. The compound of claim 18, wherein R⁴ is unsubstituted aralkyl.

21. The compound of claim 8, wherein the compound is selected from: 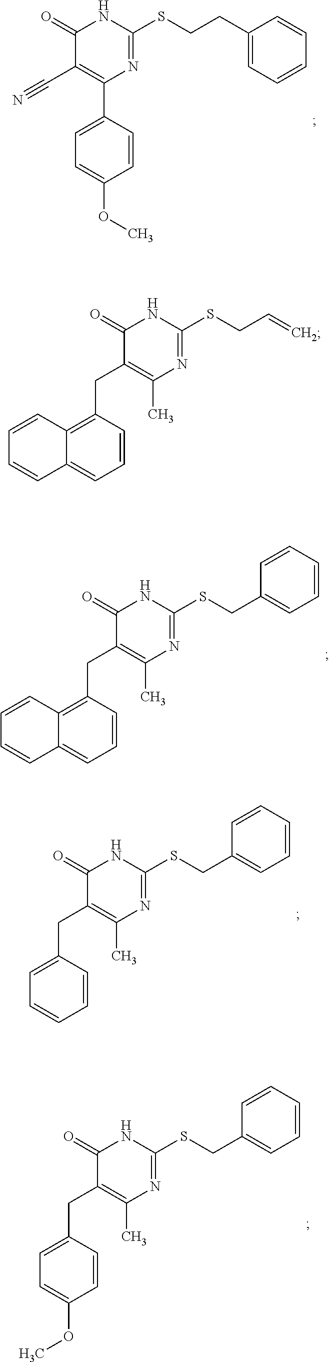 [SMILES: COc1ccc(-c2nc(SCCc3ccccc3)[nH]c(=O)c2C#N)cc1 ; C=CCSc1nc(C)c(Cc2cccc3ccccc23)c(=O)[nH]1 ; Cc1nc(SCc2ccccc2)[nH]c(=O)c1Cc1cccc2ccccc12 ; Cc1nc(SCc2ccccc2)[nH]c(=O)c1Cc1ccccc1 ; COc1ccc(Cc2c(C)nc(SCc3ccccc3)[nH]c2=O)cc1] 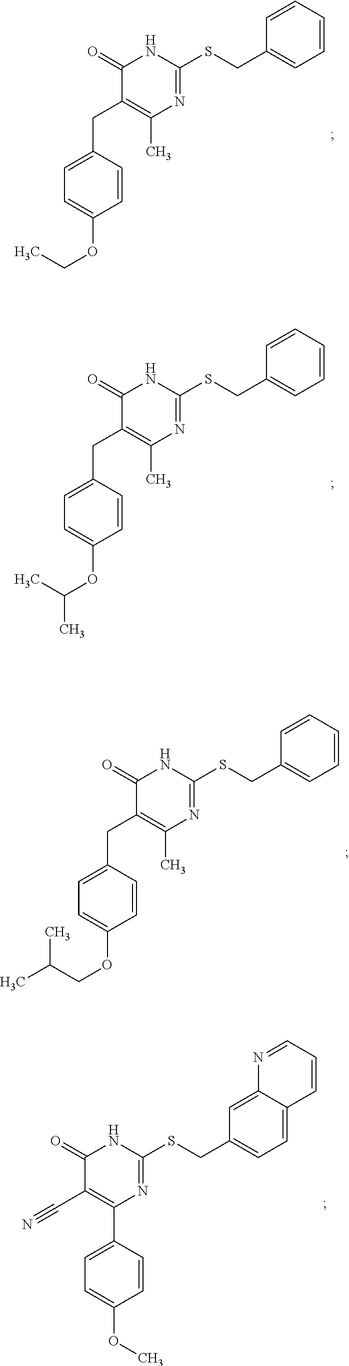 [SMILES: CCOc1ccc(Cc2c(C)nc(SCc3ccccc3)[nH]c2=O)cc1 ; Cc1nc(SCc2ccccc2)[nH]c(=O)c1Cc1ccc(OC(C)C)cc1 ; Cc1nc(SCc2ccccc2)[nH]c(=O)c1Cc1ccc(OCC(C)C)cc1 ; COc1ccc(-c2nc(SCc3ccc4cccnc4c3)[nH]c(=O)c2C#N)cc1] 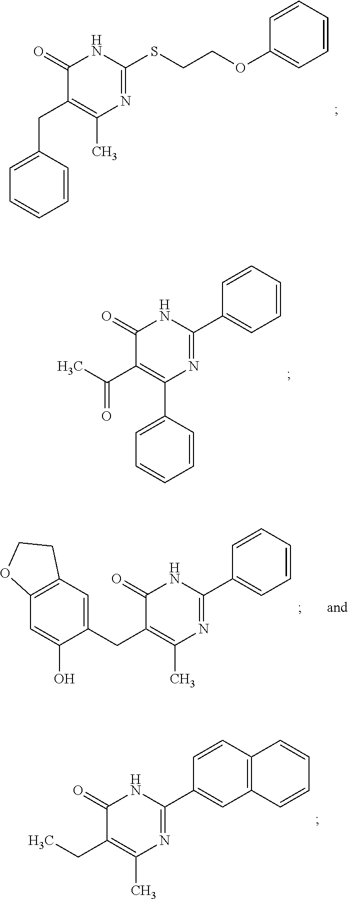 [SMILES: Cc1nc(SCCOc2ccccc2)[nH]c(=O)c1Cc1ccccc1 ; CC(=O)c1c(-c2ccccc2)nc(-c2ccccc2)[nH]c1=O ; Cc1nc(-c2ccccc2)[nH]c(=O)c1Cc1cc2c(cc1O)OCC2 ; CCc1c(C)nc(-c2ccc3ccccc3c2)[nH]c1=O]
or a pharmaceutically acceptable salt thereof.

22. A pharmaceutical composition comprising the compound of claim 1, or a pharmaceutically acceptable salt thereof, and a pharmaceutically acceptable carrier or excipient.

23. A method for modulating neutral sphingomyelinase 2 (n-SMase2) in a cell, comprising contacting a cell with a compound of claim 1 or a pharmaceutically acceptable salt thereof.

24-26. (canceled)

27. A method for inhibiting the spread of Tau seeds from donor cells to recipient cells, comprising contacting the cells with a compound of claim 1 or a pharmaceutically acceptable salt thereof.

28-30. (canceled)

31. A method for treating or preventing a disease or disorder associated with accumulation and/or aggregation of misfolded proteins, comprising administering to a subject in need thereof a compound of claim 1 or a pharmaceutically acceptable salt thereof.

32. (canceled)

33. (canceled)

34. A method for treating or preventing a neurodegenerative disease or disorder, comprising administering to a subject in need thereof a compound of claim 1 or a pharmaceutically acceptable salt thereof.

35. (canceled)
